SEQUENCE LISTING (1 sequence)

SEQ ID NO: 1 - protein, 10 aa, Artificial Sequence
  Predicted I-A bm12-specific peptide capable of binding H-2kd
EYWNSQPEFL

(19) United States
(12) Patent Application Publication
(10) Pub. No.: US 2014/0235543 A1
(43) Pub. Date: Aug. 21, 2014

(54) METHODS FOR ISOLATING AND USING A SUBSET OF CD8 T-CELLS THAT ARE RESISTANT TO INHIBITORS

(71) Applicant: Raymond M. Johnson, Indianapolis, IN (US)

(72) Inventor: Raymond M. Johnson, Indianapolis, IN (US)

(21) Appl. No.: 14/181,327

(22) Filed: Feb. 14, 2014

Related U.S. Application Data
(63) Continuation-in-part of application No. 13/811,806, filed on Apr. 8, 2013.
(60) Provisional application No. 61/764,565, filed on Feb. 14, 2013.

(51) Int. Cl. G01N 33/569

(52) U.S. Cl. CPC G01N 33/56966
USPC 514/16.5; 514/291; 435/7.21

(57) ABSTRACT

Methods for identifying a subset of CD8 T cells that is resistant to an inhibitor, specifically cyclosporine, rapamycin and/or tacrolimus, by detecting expression levels of human biomarkers are disclosed. The methods include determining whether a subset of certain CD8 T cells expresses elevated levels of Scin and/or Pla2g4a, with an elevated level indicative of proliferation of the identified CD8 T cells. Also disclosed are methods of diagnosing, monitoring and treating rheumatoid arthritis and transplant rejection, including determining and/or monitoring the expansion of a subset of CD8 T cells by measuring the level of expression of a biomarker in the population of the CD8 T cell subset.

### PRIORITY CLAIM

This application is related to (a) and claims the priority benefit of U.S. Provisional Patent Application Ser. No. 61/764,565 to Johnson, filed Feb. 14, 2013; and (b) co-pending U.S. patent application Ser. No. 13/811,806 to Johnson, filed Apr. 8, 2013, which is related and claims the priority benefit of, International Application PCT/US11/45212, filed Jul. 25, 2011, which is related to and claims the priority benefit of U.S. provisional patent application No. 61/367,127 filed on Jul. 23, 2010. The entire contents of the aforementioned priority and related applications are hereby incorporated by reference in their entirety into this disclosure.

### STATEMENT REGARDING FEDERALLY SPONSORED RESEARCH OR DEVELOPMENT

This invention was made with government support under NIH-K08-A1052128-01 awarded by the National Institutes of Health. The United States Government has certain rights in the invention.

### BACKGROUND

Current surgical techniques and the use of immunosuppressive medications have significantly improved the survival of solid organ transplant patients. One year survival rates are currently >80% and, as such, acute rejection is no longer a major cause of graft loss and transplant patient death. However, chronic allograft rejection continues to pose a serious threat to the long-term survival of transplant patients as current immunosuppressive strategies fail to significantly reduce the risk of chronic rejection.

Chronic allograft rejection has surpassed acute rejection as the leading cause of morbidity/morality in solid organ and bone marrow transplant patients. For example, around 50% of lung and heart transplant patients have graft attrition at 5 & 10 years post transplant respectively (1). Furthermore, roughly 50% of lung transplant patients develop bronchiolitis obliterans syndrome (BOS) by year five (2), while about 50% of surviving cardiac transplant patients exhibit chronic allograft vasculopathy (CAV) by year 8 (3). Chronic allograft rejection similarly plagues the long term survival of bone marrow transplant patients, with the frequency of chronic graft versus host disease in allogeneic bone marrow transplantation sitting around 50% at year two (4).

In solid organ transplants, chronic allograft rejection is characterized by progressive fibrosis and intimal proliferation rather than the acute inflammation and necrosis seen in acute rejection. Given the distinct pathologies associated with chronic allograft rejection and CAV, there is a need to better understand the pathophysiology of chronic allograft rejection. The mechanisms underlying chronic rejection are not well understood and have not been systematically investigated in human models. For example, the identity of chronic allograft rejection T cell subsets resistant to cyclosporine and/or rapamycin therapy and the T cell receptor (TCR) signalling pathways associated with resistance to current immunosuppressive regimens are uncertain. Understanding the pathophysiology of chronic rejection is critical toward developing new strategies for therapeutic interventions to improve long term survival in transplant patients.

There are mouse models for BOS based on heterotopic tracheal transplantation (5) and bone marrow transplantation (6). Data from the heterotopic trachea transplant model have shown that allogeneic airway epithelial cells are the primary target of the T cell response (7), and that, absent immunosuppression, both CD4 and CD8 T cells can mediate rejection (8). A recent rat orthotopic lung allo-transplant model incorporating cyclosporine A (CsA) and rapamycin treatment reproduced the histopathology of BOS (9); similarly an additional study showed that rapamycin was ineffective in preventing CAV in a rat cardiac transplant model (10). The animal model data are consistent with the clinical experience that even the continuous administration of current immunosuppressive drugs, including mTOR inhibitors (11), may not effectively inhibit T cell subsets mediating chronic rejection.

Intensive investigations in murine CAV models have provided insight into the effector T cell subset mediating chronic allograft rejection. In one mouse CAV model, primed CD8 T cells were sufficient to cause vasculopathy in completely MHC-mismatched aortic grafts in mice treated with cyclosporine. Intimal proliferation was independent of allo-MHC class I on the aortic graft, implying CD8 recognition of allo-MHC class II molecules (12). Similarly in a nude mouse model, adoptive transfer of naïve CD8 T cells was sufficient to cause CAV in MHC class II-mismatched bm12 cardiac allografts, again implying CD8 recognition of allo-MHC class II molecules. In that study CAV was dependent on IFN-γ, but not perforin or fas ligand (13). In an additional noteworthy study, CsA prevented vasculopathy caused by CD4 T cells, but was ineffective in preventing vasculopathy caused by CD8 T cells (14). Accordingly, effector CD8 T cells rather than effector CD4 T cells appear to have a calcineurin-independent pathway that results in T cell activation during chronic allograft rejection. Furthermore, conventional literature supports a central role for allogeneic epithelial cells as primary targets for chronic allograft rejection, and non-cytolytic CD8 T cells making IFN-γ as effectors of chronic allograft rejection in the presence of calcineurin inhibitors.

### BRIEF DESCRIPTION OF THE SEQUENCE LISTING

SEQ ID NO. 1 is a predicted I-A^{bm12}-specific peptide which, according to the subject disclosure, is predicted to bind H-2K^{d}.

### REFERENCE TO SEQUENCE LISTING SUBMITTED VIA EFS-WEB

This application is being filed electronically via EFS-WEB and includes an electronically submitted sequence listing in .txt format. The .txt file contains a sequence listing entitled “D201402USCIP” created on Feb. 14, 2014, and is 4,096 bytes in size. The information recorded in this .txt file is identical to the written sequence listing contained in this disclosure and is hereby incorporated by reference herein in its entirety.

### BRIEF SUMMARY

Some aspects of the invention include methods for identifying a subset of CD8 T cells that is resistant to an inhibitor, the method comprising the step of determining whether the CD8 T cells express an elevated level of at least one biomarker selected from the group consisting of Scin and Pla2g4a, and wherein the existence of the elevated level of the at least one biomarker is indicative of the proliferation of the CD8 T cells within the subset. The inhibitor may be selected from the group consisting of cyclosporine, rapamycin and tacrolimus. In at least one exemplary embodiment, the CD8 T cells are mammalian. In yet another exemplary embodiment, the at least one biomarker of the method is a human biomarker.

Additional methods for diagnosing a condition in an individual are disclosed. In at least one exemplary embodiment, the method comprises the step of determining if the individual has experienced an expansion of a subset of CD8 T cells that are resistant to an inhibitor selected from the group consisting of cyclosporine, tacrolimus and rapamycin. The step of determining if the individual has experienced an expansion of a subset of CD8 T cells may comprise measuring the level of expression of at least one biomarker selected from the group consisting of Scin and Pla2g4a in a population of the subset of CD8 T cells from the individual and determining if such level of expression is elevated. Additionally, in at least one embodiment, the at least one biomarker comprises a human biomarker. In certain embodiments of the methods disclosed herein the condition may comprise rheumatoid arthritis and in alternative exemplary embodiments, the condition may comprise allograft rejection.

Additional embodiments disclosed herein comprise methods for treating a condition in an individual comprising the step of administering at least one therapeutically effective dose of at least one compound to a patient capable of inhibiting proliferation of a subset of CD8 T cells from the individual that express AHR and that are resistant to an inhibitor selected from the group consisting of a calcineurin inhibitor and a mTOR inhibitor, wherein the at least one compound comprises a calcineurin inhibitor and an AHR hydrocarbon inhibitor. In at least one exemplary embodiment, the condition comprises rheumatoid arthritis and the step of administering is performed to treat the individual experiencing rheumatoid arthritis. In yet another exemplary embodiment, the condition comprises transplant rejection and the step of administering at least one therapeutically effective dose is performed to treat the individual experiencing allograft vasculopathy or chronic allograft rejection including, but not limited to, the condition of graft versus host disease. The subset of CD8 T cells may comprise CD8bm12-1 or an equivalent cell type and, in at least one embodiment, the at least one biomarker comprises a human biomarker.

Alternatively or additionally, the method may further comprise the step of monitoring the development of the condition in the individual by determining if the individual has experienced an expansion of the subset of CD8 T cells that are resistant to an inhibitor selected from the group consisting of cyclosporine, tacrolimus and rapamycin. Furthermore, the step of determining if the individual has experienced an expansion of the subset of CD8 T cells may further comprise the step of determining whether the individual has elevated expression of at least one biomarker selected from Scin or Plag2g4a in a population of the subset of CD8 T cells, and wherein the existence of elevated expression of the at least one biomarker is indicative of the proliferation and expansion of the CD8 T cells.

Additional methods are disclosed for monitoring the treatment of a condition in an individual. Embodiments of such methods comprise the step of determining if the individual has experienced an expansion of a subset of CD8 T cells that are resistant to an inhibitor selected from a group consisting of cyclosporine, tacrolimus and rapamycin. Additionally, such methods may further comprise the step of determining whether the individual has elevated expression of at least one biomarker selected from a group consisting of Scin and Plag2g4a in a population of the subset of CD8 T cells, and wherein the existence of elevated expression of the at least one biomarker is indicative of the proliferation and expansion of that subset of CD8 T cells. In certain non-limiting embodiments, the condition may comprise rheumatoid arthritis. In such embodiments, the absence of elevated expression of the at least one biomarker may be indicative of effective treatment of the rheumatoid arthritis. In other embodiments, the condition may comprise allograft rejection.

Methods are also disclosed for predicting the susceptibility of an individual to transplant rejection. In certain embodiments, a method for predicting the susceptibility of an individual to transplant rejection comprises the step of determining whether the individual has elevated expression of at least one biomarker selected from Scin or Plag2g4a in a population of CD8 T cells from the individual, wherein the absence or existence of elevated expression of the at least one biomarker is indicative that the individual is more or less susceptible to transplant rejection. Such biomarker may comprise a human or an otherwise mammalian biomarker. Additionally, the method may further comprise the step of determining if the individual has expansion of a subset of CD8 T cells that are resistant to an inhibitor selected from the group consisting of cyclosporine, tacrolimus and rapamycin. In embodiments of such a method, an expansion of the subset of CD8 T cells is indicative of transplant rejection.

Additional methods are disclosed for monitoring the treatment of a condition in an individual by determining if the individual has experienced an expansion of a subset of CD8 T cells that are resistant to an inhibitor selected from the group consisting of cyclosporine, tacrolimus and rapamycin. Additionally, such methods may further comprise the step of determining whether the individual has elevated expression of at least one biomarker selected from the group consisting of Scin and Plag2g4a in a population of the subset of CD8 T cells from the individual and wherein the existence of elevated expression of the at least one biomarker is indicative of the proliferation of the CD8 T cells. In certain non-limiting embodiments, the condition may comprise rheumatoid arthritis. In such embodiments, the absence of elevated expression of the at least one biomarker may be indicative of effective treatment of the rheumatoid arthritis. In other embodiments, the condition may comprise an allograft transplant or allograft rejection. In such embodiments, the absence of elevated expression of the at least one biomarker may be indicative of effective treatment of allograft rejection.

Some aspects of the invention include methods for isolating a subset of CD8 T-cells, comprising the steps of: transplanting an allorgan into an animal and monitoring the animal; recovering CD8 T-cells from the animal, culturing said CD8 T-cells on a layer of semi-professional antigen presenting cells; and selecting the lymphocyte cells that proliferate on the layer of semi-professional antigen presenting cells. In some embodiments the semi-professional antigen presenting cells are selected from the group consisting of smooth muscle cells, endothelial cells and epithelial cells. Some embodiments further include the steps of: contacting said CD8 T-cells with cyclosporine, wherein the concentration of cyclosporin in contact with said cells is sufficient to inhibit the growth of most types of CD8 T-cells; and harvesting the CD8 T-cells that proliferate in the presence of cyclosporin.

Some methods for isolating a subset of CD8 T-cells further include the steps of contacting said CD8 T cells with an antibody wherein the antibody selectively binds to IL-18r1. And still other embodiments include the steps of testing said CD8 T-cells to determine if the CD8 T-cells express at least one gene selected from the group consisting of: Mest, Padi2, Ahr, Klh16, Rasgrp3, Klhi30, Trib2, Rab17, Prkcz, PLCγ2, scin, Pla2g4a, CD7, Il18r1, IL-17a, IL-17f, Sgk3, Gpr15, Pls3 and Zfp187. In some embodiments the CD8 T-cell is CD8bm12-1 or an equivalent cell type. The proliferation and/or viability of these cells may be effected by contacting them with compounds that interact with the aryl hydrocarbon receptor or components of the novel CD8bm12-1 T cell receptor signalling cascade.

Other aspects of the invention include methods of collecting cyclosporin and/or rapamycin resistant CD8 T-cells, comprising the steps of: providing a sample from an animal wherein the sample includes CD8 T-cells, wherein the animal has undergone an allorgan transplant, supplying an antibody, wherein said antibody selectively binds to at least one protein selected from the group consisting of anti-CD7 antibody or anti-Il-18r1 antibody; contacting the sample with said antibody.

Still other aspects of the invention include methods for collecting cyclosporin and/or rapamycin resistant CD8 T-cells, comprising the steps of: providing a sample from an animal wherein the sample includes CD8 T-cells, wherein the animal has undergone an allorgan transplant; supplying an antibody, wherein said antibody selectively binds to Il18r1; and contacting the sample with said antibody. In some embodiments the method further includes the step of: recovering the CD8-T cells that were contacted with said antibody to Il18r1 and wherein the CD8 T-cells that are recovered are bound to an antibody to Il18r1. In some embodiments the CD8 T-cell is CD8bm12-1 or an equivalent cell type.

Yet other aspects of the invention include methods for selecting for compounds that regulate a subset of CD8 T-cells which are resistant to cyclosporin and/or rapamycin, comprising the steps of: providing a population of CD8 T-cells wherein said CD8 T-cells express Il18r1 and aryl hydrocarbon receptor (Ahr); contacting said CD8 T-cells with at least one compound; and measuring the effect of the compounds on the population of said CD8 T-cells. The CD8 T-cells may be contacted with the compound either in vitro or in vivo. Compounds that help to regulate this subset of CD8 T-cells may act, at least in part, by inhibiting the expression or function of at least one of the genes selected from the group consisting of: Mest, Padi2, Ahr, Klh16, Rasgrp3, Klhi30, Trib2, Rab17, Prkcz, PLCγ2, scin, Pla2g4a, CD7, Il18r1, IL-17a, IL-17f, Sgk3, Gpr15, Pls3 and Zfp187. In some embodiments the compounds are selected from a group of compounds that preferentially binds to at least one gene product encoded by at least one of the genes selected from the group consisting of: Mest, Padi2, Ahr, Klh16, Rasgrp3, Klhi30, Trib2, Rab17, Prkcz, PLCγ2, scin, Pla2g4a, CD7, Il18r1, IL-17a, IL-17f, Sgk3, Gpr15, Pls3 and Zfp187.

Other aspects of the invention include methods for treating allograft rejection, comprising the steps of: identifying a patient wherein said patient has undergone an allorgan transplant; providing at least one therapeutically effective dose of at least one compound that inhibits the proliferation of a subset of CD8 T-cells, wherein said subset of CD8 T-cells expresses Il18r1 and/or the Ahr and is resistant to levels of cyclosporin that inhibit most other subsets of CD8 T-cells; and treating the patient with the at least one therapeutically effective dose of the at least one compound. In some embodiments the patient is either a human or an animal. In some embodiments the compound that is used to effect the activity of the subset of CD8 T-cells is a small molecule that inhibits the expression of at least one gene selected from the group consisting of: Mest, Padi2, Ahr, Klh16, Rasgrp3, Klhi30, Trib2, Rab17, Prkcz, PLCγ2, scin, Pla2g4a, CD7, Il18r1, IL-17a, IL-17f, Sgk3, Gpr15, Pls3 and Zfp187. While in still other embodiments the compound is an interference RNA. And in other embodiments the compound is at least one selected compound selected from the group consisting of; polyclonal antibodies, or monoclonal antibodies, wherein the antibodies preferentially bind to the cyclosporin resistant CD8 T-cells. In still another embodiment the compound is humanized anti CD7 antibody. While in still another embodiment the compound is humanized anti IL-18 receptor antibody.

Still other aspects of the invention include methods of inhibiting or at least reducing the progression of chronic allograft rejection comprising the steps of contacting cyclosporin resistant CD8 T-cells with at least one compounds that inhibits the proliferation of this subset of cells, compounds suitable for inhibiting these cells include compounds that inhibit at least one gene product selected from the group consisting of antibodies such as anti-CD7 antibody, small molecules and the like.

In some embodiments the compound used to regulate chronic allograft rejection is an antibody that preferentially binds to CD7 such antibodies may include, but are not limited to, the human mouse chimeric CD7 monoclonal antibody SDZCHH380. Still other embodiments include methods of selecting for at least one molecule that inhibits or at least slows the progression of chronic allograft rejection, comprising the step of providing at a population of cyclosporin resistant CD8 T-cells; contacting the cells with at least one compound and measuring the compound's effect on the proliferation and or survival of said cyclosporin resistant CD8 T-cells.

Some embodiments include methods for treating allograft vasculopathy or chronic allograft rejection, comprising the steps of: identifying a patient that has undergone an allorgan or an allograft transplant; providing at least one therapeutically effective dose of at least one compound that inhibits the proliferation of a subset of CD8 T-cells, wherein said subset of CD8 T-cells expresses Il18r1 and is resistant to levels of cyclosporin and/or rapamycin that inhibits the proliferation of most other subsets of CD8 T-cells; and treating the patient with the at least one therapeutically effective dose of said at least one compound. In some embodiments the patient treated for allograph vaculopathy is either an animal or a human.

In some embodiments that compounds used to treat the patient is as leas one compound that inhibits the expression or function of at least one gene selected from the group consisting of: Mest, Padi2, Ahr, Klh16, Rasgrp3, Klhi30, Trib2, Rab17, Prkcz, PLCγ2, scin, Pla2g4a, CD7, Il18r1, IL-17a, IL-17f, Sgk3, Gpr15, Pls3 and Zfp187 or the function of a least one gene product encoded by at least one gene selected from the group. In some embodiments compound is selected from a group of compounds consisting of; polyclonal antibodies, or monoclonal antibodies, wherein the antibodies preferentially bind to the cyclosporin-resistant CD8 T-cells. In some embodiments the compound is at least one compound selected from the group consisting of either humanized or non-humanized anti CD7 antibody and humanized or non-humanized anti IL-18 receptor antibody. And in still other embodiments the compound is a siRNA that alters the expression of at least one gene in the cyclosporin resistant CD8 T-cells.

Some embodiments are methods for diagnosing a medical condition, comprising the steps of: analyzing a sample from a patient for the presence of a population of CD8 T-cells that are resistant to levels of cyclosporin and/or rapamycin that inhibits the proliferation of most other subsets of CD8 T-cells. In some embodiments the diagnostic methods include the step of determining if said population of CD8 T-cell in the sample express the IL-18 receptor. In some embodiments the diagnostic method includes: contacting a population of CD8 T-cells with at least one antibody that binds to least one of the following: the IL-18 receptor, and the aryl-hydrocarbon receptor with said CD8 T-cells. In some embodiments CD8 T-cells are cultured in the presence of levels of cyclosporin and or rapamycin that would inhibit the proliferation of most types of CD8 T-cells.

Some embodiments include systems for diagnosing a medical condition; comprising; a sample from a patient, wherein the sample includes CD8 T-cells; a set of conditions for growing the cells in vitro in the presence of levels of cyclosporin and/or rapamycin that inhibit the growth of most types of CD8 T-cells; and an assay for determining if there is a population of CD8 T-cells in the sample that proliferate in the presence of levels of cyclosporin and/or rapamycin that inhibit the growth of most types of CD8 T-cells. In some embodiments the system further includes at least one reagent wherein the reagent binds to an IL-18 receptor on the surface of a subset of CD8 T-cells in the sample or to an aryl hydrocarbon receptor in said subset of CD8 T-cells. In some methods or systems for diagnosing disease sample that includes CD8 T-cells are placed in contact with at least one compound that selectively or at least preferentially interacts with at least one gene product produced by at least one gene selected from the group consisting of: Mest, Padi2, Ahr, Klh16, Rasgrp3, Klhi30, Trib2, Rab17, Prkcz, PLCγ2, scin, Pla2g4a, CD7, Il18r1, IL-17a, IL-17f, Sgk3, Gpr15, Pls3 and Zfp187.

In some embodiments reagents that bind to elements of CD8 T-cells that are resistant to cyclosporin or rapamycin are bound to solid surface such as a bead or a column; this property may be used to help recover and/or identify CD8 T-cells that are resistant to cyclosporin and/or rapamycin. In some embodiments the compounds that interact with CD8 T-cells that are resistant to cyclosporin and/or rapamycin are themselves label with a moiety that can be used to detect them. Such moieties include, but are not limited to, radioisotopes, fluorophores and chemiluminescent groups.

Some embodiments include methods and or systems for diagnosing diseases or conditions in humans or animals that include determining if CD8 T-cells in a sample from the patient expresses at least one gene selected from the group Mest, Padi2, Ahr, Klh16, Rasgrp3, Klhi30, Trib2, Rab17, Prkcz, PLCγ2, scin, Pla2g4a, CD7, Il18r1, IL-17a, IL-17f, Sgk3, Gpr15, Pls3 and Zfp187. In some embodiments the CD8 T-cells are assayed for combination of these genes or gene products. Some embodiments include identifying the CD8-T-cells as being resistant to cyclosporin and/or rapamycin. Some of the assays for gene products may include the use of labelling reagent and techniques such as flow-cytometry. Some of the assays for gene expression may include the use of RT-PCR which may be combined with reverse transcription to quantify the level of mRNA from a given gene.

### BRIEF DESCRIPTION OF THE FIGURES

FIG. 1 illustrates a flow cytometry of CD4 vs. CD8 on polyclonal T cell populations from mouse #2 and mouse #3 after culturing immune splenocytes from C57BL/6 mice (1-A^{b}) previously primed with bm12 skin grafts on Bm12.4 epithelial cells (I-A^{bm12}; MHC class II mismatch) as the alloantigen presenting cell; result: yielded exclusively CD8 T cells rather than the conventional CD4 T cells that were expected in the MHC class II-mismatched bm12 mouse model.

FIG. 2A shows a bar graphs of pg ml⁻¹ of IFN-gamma produced by CD8 alloreactive T-cell clones recognizing either Bm1.11 (white bars) or Bm12.4 (black bars); *=p value<0.001 and demonstrates the specificity of the T cell clones.

FIG. 2B shows bar graphs of pg ml⁻¹ of IFN-gamma produced by CD8 alloreactive T-cell clones in media (hatched bar), or activated by CL.7 control cells (white bars) or CL.7I -A^{bm12} cells (black bars); *=p value<0.001 and demonstrates that CD8bm12-1 & CD8bm12-2 recognize MHC class II I-A^{bm12} directly.

FIG. 3A shows flow cytometry data measuring cell surface phenotypes of CD8bm1.

FIG. 3B shows flow cytometry data measuring cell surface phenotypes of CD8bm12-1.

FIG. 3C shows flow cytometry data measuring cell surface phenotypes of CD8bm12-2.

FIG. 4A shows a plot of % specific lysis, vs. Effector target ratio as measured for CD8bm1, CD8bm12-1, and CD8bm12-2 with allo-epithelial target cells (Bm1.11 for CD8bm1; Bm12.4 for CD8bm12-1 & CD8bm12-2) and demonstrates that the novel CD8 T cell clones CD8bm12-1 & CD8bm12-2 recognizing MHC class II I-A^{bm12} have a non- or minimally-cytolytic phenotype.

FIG. 4B shows a plot of % specific lysis vs. Effector:Target ratio measured for two fibroblast-derived alloreactive CD8 T cell clones with H-2^{d} bearing CL.7 fibroblast targets and demonstrates cytolytic capabilities of conventional CD8 T cells.

FIG. 5A shows a plot of proliferation of CD8bm1 (▪) and CD8bm12-1 () activated by immobilized anti-CD3 over the full range of cyclosporine A tested in Experiment #1.

FIG. 5B shows a plot of proliferation of CD8bm1 (▪) and CD8bm12-1 () activated by immobilized anti-CD3 over the full range of cyclosporine A tested in Experiment #2.

FIG. 6A shows a plot of IL-2 production of CD8bm1 (▪) and CD8bm12-1 () activated by immobilized anti-CD3 over the full range of cyclosporine A tested in Experiment #2 of FIG. 5B.

FIG. 6B shows a plot of IL-10 production of CD8bm1 (▪) and CD8bm12-1 () activated by immobilized anti-CD3 over the full range of cyclosporine A tested in Experiment #2 of FIG. 5B.

FIG. 6C shows a plot of IFN-gamma production of CD8bm1 (▪) and CD8bm12-1 () activated by immobilized anti-CD3 over the full range of cyclosporine A tested in Experiment #2 of FIG. 5B.

FIG. 7A shows a plot of the proliferation of CD8bm12-1 cells exposed to only media (unactivated cells), and CD8bm12-1 cells activated by exposure to immobilized anti-CD3 antibody in the presence of 30 μM, 10 μM, 3 μM, or 0 μM CH-223191 (aryl hydrocarbon receptor antagonist). **=pvalue<0.005; ***=pvalue<0.0005.

FIG. 7B shows a plot of the proliferation of CD8bm12-1 cells exposed to only media (unactivated cells), and CD8bm12-1 cells in the presence of 1 μg/ml CSA activated by exposure to immobilized anti-CD3 antibody in the presence of 30 μM, 10 μM, 3 μM, or 0 μM CH-223191. **=pvalue<0.005; ***=pvalue<0.0005

FIG. 8 shows a plot of the proliferation of CD8bm12-1 cells exposed to only media (unactivated cells), and CD8bm12-1 cells activated by exposure to immobilized anti-CD3 antibody in the presence of 25 ηM rapamycin. ***=pvalue<0.0005.

FIG. 9 shows a bar graph evidencing that CD8bm12-1, but not CD8bm1, produced IL-17 when activated by immobilized anti-CD3 antibody.

FIG. 10 shows bar graphs depicting data collected from CD8bm12-1 T cells activated by immobilized anti-CD3 in the absence and presence of CSA and the absence and presence of varied concentrations of the AHR inhibitor CH-223191 (μM). The specific data relates to: A) proliferation in media without CsA; B) proliferation in the presence of 1 μg/ml CsA. Aggregate data from two independent experiments; **=pvalue<0.005; ***=pvalue<0.0005. Proliferation for the unactivated condition (proliferation in media without immobilized anti-CD3) is subtracted from the matched activated (with anti-CD3) condition in order to evaluate on proliferation driven by T cell receptor activation.

FIG. 11 shows bar graphs depicting data collected from CD8bm12-1 T cells activated by immobilized anti-CD3/CD28 in the absence and presence of varied concentrations of the AHR inhibitor CH-223191 (μM) and rapamycin (ηM). The specific data relates to: A) proliferation in media without CsA; B) proliferation in the presence of 1 μg/ml CsA; C) IL-17 levels in condition shown in panel A; and D) IFN-γ levels in condition shown in panel A. Aggregate data from two independent experiments; **=pvalue<0.005; ***=pvalue<0.0005. Proliferation for the unactivated condition (proliferation in media without immobilized anti-CD3) is subtracted from the matched activated (with anti-CD3) condition in order to evaluate on proliferation driven by T cell receptor activation.

FIG. 12 shows bar graphs depicting data that supports the AHR inhibitor CH-223191 has minimal inhibitory effect on homeostatic proliferation driven by IL-7, while rapamycin significantly inhibits IL-7 driven homeostatic proliferation. *=pvalue<0.05; **=pvalue<0.005; ***=pvalue<0.0005.

FIG. 13 shows confirmation of CD8bm1 and CD8bm12-1 T cell microarray gene expression patterns when activated by anti-CD3 in the presence of 1 μg/ml CsA using conventional RT-PCR. An inverted image of an ethidium bromide stained gel is shown for presentation purposes.

FIG. 14 shows differential levels of Pla2g4a and Scin in total RNA isolated from the circulating CD8 T cell pool of the five subjects by RT-PCR: 1) a healthy control subject, 2) an early renal transplant patient (second transplant) with a 4 ml/min decline in CrCl, 3) a late renal transplant patient approaching dialysis with no change in CrCl, 4) a liver transplant patient (due to Hepatitis C virus infection) 5 years post transplant, and 5) a patient with CCP-positive rheumatoid arthritis. An inverted image of an ethidium bromide stained gel is shown for presentation purposes.

FIGS. 15A-15C show representative micrographs of stained lung sections from subject mice and support that adoptive transfer of CD8bm12-1 causes bronchiolitis obliterans syndrome (BOS) in bm12 mice.

### DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended. Indeed, methods of the present disclosure may be embodied in many different forms and should not be construed as being limited to the embodiments expressly set forth herein.

Similarly, many modifications and other embodiments of the methods described herein will come to mind to one of skill in the art to which the present disclosure pertains having had the benefit of the teachings presented in the present descriptions and associated drawings. Therefore, it is to be understood that any such alterations, modifications, and further applications of the principles of the present disclosure are intended to be included within the scope of the appended claims. Furthermore, unless otherwise defined, all technical and scientific terms used herein have the same meaning as commonly understood by one of skill in the art to which the present disclosure pertains.

The disclosure of the present application provides various methods relating to the identification of the pathways behind and the detection and prevention of chronic allograft rejection. Specifically, certain embodiments of the methods disclosed herein facilitate the prevention of chronic allograft rejection by identifying a novel cyclosporine- and/or rapamycin-resistant TCR signalling pathway that is dependent on the aryl hydrocarbon receptor and, specifically, by using one or more human biomarkers to identify elevated levels of cyclosporine- and/or rapamycin-resistant CD8 T cell subsets. Furthermore, various methods are disclosed for diagnosing, treating, and monitoring certain medical conditions in humans, including rheumatoid arthritis and chronic allograft rejection.

Unless specifically stated otherwise the term, ‘therapeutically effective dose,’ as used herein includes an amount of a compound that administered either one time or over the course of treatment cycle affects the health, wellbeing or mortality of a human or animal patient.

Unless specifically stated otherwise the term, ‘semi-professional antigen presenting cells’ or ‘semi-professional APC’ as used herein refers to non-bone marrow derived cells with low basal levels of MHC class II expression that can be increased with exposure to IFN-gamma (15).

Unless specifically stated otherwise, the term “about” refers to a range of values plus or minus 10%, for example, for percentages and plus or minus 1.0 unit for unit values, for example about 1.0 refers to a range of values from 0.9 to 1.1.

Cyclosporine may also include salts of compounds having the formula (E)-14,17,26,32-tetrabutyl-5-ethyl-8-(1-hydroxy-2-methylhex-4-enyl)-1,3,9,12,15,18,20,23,27-nonamethyl-11,29-dipropyl-1,3,6,9,12,15,18,21,24,27,30-undecaazacyclodotriacontan-2,4,7,10,13,16,19,22,25,28,31-undecaone, although other related molecules may also be used to practice some aspects of the invention. The terms, ‘Cyclosporin’ and ‘Cyclosporine’ may both used as neither appears to be the preferred term of art in the literature.

Rapamycin may also include salts of compounds having the formula (3S,6R,7E,9R,10R,12R,14S,15E,17E,19E,21S,23S,26R,27R,34aS)-9,10,12,13,14,21, 22,23,24,25,26,27,32,33,34,34a-hexadecahydro-9,27-dihydroxy-3-[(1R)-2-[(1S,3R,4R)-4-hydroxy-3-methoxycyclohexyl]-1-methylethyl]-10,21-dimethoxy-6,8,12,14,20,26-hexamethyl-23 ,27-epoxy-3H-pyrido[2,1-c][1,4]-oxaazacyclohentriacontine-1,5,11,28,29 (4H,6H,31H)-pentone.

The term ‘collect’ used in reference to certain cell types includes cell populations that have been enriched in a particular type of cells. Enriched cell populations may include more cells of a particular type of cell in the population than are found in healthy physiological sources.

Chronic allograft rejection is the major cause of organ failure and death in solid organ transplant patients. In cardiac transplantation, conventional interventions that focus on atherosclerosis pathogenesis do not prevent cardiac allograft vasculopathy (CAV). Indeed, chronic allograft rejection progresses in spite of the administration of calcineurin inhibitors or mTOR inhibitors, other metabolic inhibitors of lymphocytes, or glucocorticoid therapy. Animal models have clearly shown that chronic rejection vasculopathy is T cell dependent and involves both CD4 and CD8 T cell subsets. The unusual histopathology (intimal proliferation and fibrosis instead of tissue necrosis) and resistance to calcineurin blockade, suggests that T cells driving chronic allograft rejection have an immunobiology very different from T cells studied in standard mixed lymphocyte culture systems based on professional antigen presenting cells. Specifically, T cells mediating chronic allograft rejection are likely to function in non-hematopoetic microenvironments, to have reduced cytolytic ability, and to have activation pathways independent of calcineurin and/or NFAT transcription factors (NFAT).

Clinical experience and animal models have shown that calcineurin inhibitors do not prevent CAS or BOS. The scientific literature supports a central role for allogeneic semi-professional antigen presenting cells (APC) as primary targets for chronic allograft rejection, and a role for CD8 T cells as effectors of chronic allograft rejection in the presence of calcineurin inhibitor therapies. Experimentation in murine models has demonstrated that CD8 T cell-mediated chronic rejection includes recognition of MHC class II allo-antigens, and is dependent on production of IFN-γ, but not perforin or fas ligand killing pathways. Existing data implicates a noncytolytic IFN-γ-producing CD8 T cell subset with a TCR signalling/activation pathway resistant to calcineurin inhibitors as the mediator of chronic allograft rejection.

Intensive investigations in rodent CAV models have identified the T cell subsets responsible for mediating chronic allograft rejection. It is clear that CAV is T cell-mediated because allogeneic arterial grafts transplanted into T cell deficient mice do not develop the intimal proliferative lesion characteristic of CAV (16). T cell depletion studies and knockout mice have demonstrated that CD4 T cells play a major role (16-18) and that CD8 T lymphocytes also contribute to the development of CAV (18-20). In a murine model, primed CD8 T cells were sufficient to cause vasculopathy in completely MHC-mismatched aortic grafts in mice treated with cyclosporine. In that study, intimal proliferation was also independent of allo-MHC class I expression on the aortic graft, implying CD8 recognition of allo-MHC class II molecules (12). Similarly, others showed that development of vasculopathy was dependent on CD8 T cell IFN-γ, independent of perforin, fas, and fas ligand. Naïve C57BL/6 CD8 T cells, in nude mice pre-treated with an activating anti-CD40 monoclonal antibody to up regulate the intrinsic costimulatory environment, were sufficient to cause CAV in MHC class II-mismatched bm12 cardiac allografts; again implying CD8 recognition of allo-MHC class II molecules as bm12 mice (B6.C-H-2^{bm12}) is isogenic with C57BL/6 mice except for a 3 amino acid change in the bm12 MHC II I-A^{b} beta chain. In the bm12 cardiac allograft study the development of intimal proliferation was also dependent on T cell IFN-γ, but not perforin or fas ligand (13). CAV histopathology does not resemble the tissue necrosis seen in acute rejection. Accordingly, IFN-γ production, but not potent cytolysis, is required for CD8-mediated CAV.

Various labs have identified a role for CD8 T cells in allograft vasculopathy in models using bm12 model of MHC class II mismatched vascular allografts. At first glance, CD8 recognition of allo-MHC class II molecules would appear to violate a basic cellular immunology paradigm; however T cells participating in rejection can recognize allogeneic donor graft cells via one of two basic mechanisms. ‘Direct allorecognition’ occurs when T cell receptors ‘see’ intact allogeneic major histocompatibility (MHC) molecules on donor cell surfaces; CD4 T cells ‘see’ allo-MHC II, while CD8 T cells ‘see’ allo-MHC I. ‘Indirect allorecognition’ occurs when allogeneic major or minor histocompatibility antigens are taken up by recipient APC and processed to generate an allopeptide in the context of self-MHC molecules. Peptide fragments of donor MHC class I or class II molecules can be loaded on to recipient self MHC class I or II molecules for presentation to either CD8 or CD4 alloreactive T cells respectively. Direct allorecognition is the predominant mechanism for allorecognition, and generally believed to be the principle mechanism active during acute allograft rejection, while indirect allorecognition has been associated with chronic allograft rejection (reviewed in (21, 22)). The independent research groups that reported CD8-mediated CAV in MHC class II mismatched vascular grafts postulated ‘indirect’ allo-recognition of donor MHC class II molecules. As reported herein, CD8 T cells likely directly recognize MHC class II alloantigens in the C57BL/6 versus bm12 transplant model.

The immunobiology of CD8 T cells during solid organ rejection is different than that of CD4 T cells. In patients with acute and chronic graft rejection, CD8 T cells represent a significant portion of the graft-infiltrating-lymphocytes (GIL), and likely participate in graft injury based on their activated state and ability to lyse donor-origin target cells in vitro (23-26). In animal models, CD8 T cells are resistant to costimulation blockade sufficient to induce CD4 T cell tolerance. For example, blocking CD28 costimulatory pathways with CTLA-4Ig protected intestinal allografts from CD4-mediated but not CD8-mediated rejection (27), and CD8-mediated rejection of allogeneic skin grafts was resistant to combined CD28/CD40 blockade (28).

Differential in vivo susceptibility to costimulation blockade implies that CD4 and CD8 T cells utilize different activation pathways. In human transplant patients, chronic allograft rejection and CAV is not prevented by the calcineurin inhibitors cyclosporin A or FK506 (tacrolimus) Likewise, in a murine aortic allograft vasculopathy model, CD8 T cells caused allograft vasculopathy in cyclosporin-treated mice. Accordingly, while Cyclosporin A (CsA) prevented vasculopathy caused by CD4 T cells, it was ineffective in preventing vasculopathy caused by CD8 T cells and, additionally, the CsA-resistant CD8 T cells had a phenotype that included reduced cytolytic potential (14). These results show that CD8 T cells with a non-cytolytic phenotype (rather than CD4 T cells) appear to have a calcineurin-independent pathway for T cell activation.

Furthermore, the literature supports a role for CD8 T cells in cardiac allograft vasculopathy in the setting of MHC class II mismatched grafts. Experimentation in the murine bm12 MHC class II mismatch model has demonstrated that CD8 T cell-mediated CAV is dependent on production of IFN-γ, but not the perforin- or fas ligand-mediated killing pathways, and that CD8 lymphocytes are sufficient for the development of chronic rejection (13). Likewise, as noted above, the human cardiac transplant experience shows that calcineurin inhibitors do not prevent CAV. Murine models of CAV show that cyclosporin blocks CD4-mediated, but not CD8-mediated CAV, and the murine bm12 model directly implicates CD8 T cells specific for allo-MHC class II in allograft vasculopathy. In aggregate, the existing human and animal model data imply a noncytolytic CD8 T cell subset whose activation pathway remains functional in the presence of calcineurin blockade. Histopathology in clinical specimens and rodent models show a lymphocytic endothelialitis in the vascular intima associated with CAV (29); chronic allograft rejection in other solid organs includes thickening of vascular walls and fibrosis. Accordingly, it is reasonable to postulate that T cells mediating chronic allograft rejection and CAV are being activated in the non-hematopoetic microenvironments. Immunohistochemistry of murine cardiac allografts showed expression of MHC class II molecules on endothelial cells and intimal smooth muscle cells 12 weeks post transplantation, while expression of MHC class II in control cardiac isografts was limited to epicardial macrophages (30). In the mouse bm12 model, CD8 T cell-mediated CAV is postulated to occur via an indirect pathway because of recognition of allo-MHC class II molecules. One hypothesis consistent with this data is that the MHC class II expressed on smooth muscle and endothelial cells serves as alloantigen for direct recognition by CD8 T cells in the pathogenesis of CAV.

In light of the aforementioned, embodiments of the present method utilize a novel T cell culture system based on allogeneic epithelial antigen presenting cells (semi-professional APC) to isolate a cyclosporin-resistant CD8 T cell clone with minimal cytolytic capability. Derivation of the novel alloantigen-specific CD8 T cell clones requires previous priming with an allogeneic skin graft, thus implying expansion of this T cell subset during transplant rejection. Subsequent characterization and comparison of the cyclosporin-resistant CD8 T cell clone with typical cyclosporin-sensitive CD8 T cells suggests that it is a member of a CD8 T cell subset with a unique cell surface phenotype and novel TCR activation pathways, and that these unique CD8 T cell clones reflect the immunobiology of chronic rejection within nonhematopoetic microenvironments of solid organs and vascular walls.

Methods.

Specific examples of exemplary methods and details related thereto will now be described in connection with the inventive technology. It will be appreciated that such methods and investigative details are non-limiting and provided in furtherance of promoting understanding of the disclosed novel methods and techniques.

Mice

Male BALB/c and C57BL/6 mice were obtained from Harlan Laboratories (Indianapolis, Ind.). B6.C-H2bm1/ByJ (bm1 mice) and B6.C-H2bm12/KhEg (bm12 mice) mice were obtained from Jackson Laboratories (Bar Harbor, Mass.). All mice were housed in a pathogen-free barrier animal facility in accordance with procedures and protocols approved by the Indiana University School of Medicine Animal Care & Use Committee.

Cell Lines and Culture Conditions

Previously derived cloned murine reproductive tract epithelial cell lines from bm1 (Bm1.11) and bm12 (Bm12.4) mice were maintained as previously described (31, 32). CL.7 fibroblasts (H-2d) were obtained from the American Type Culture Collection (ATCC; Manassas, Va.) were grown in DMEM media with 10% Fetal Clone III serum (Hyclone; Logan, Utah).

T Cell Clones

‘Fibroblast’ CD8 T cell clones were derived from T cell populations initially activated in mixed lymphocyte reactions (MLR). Primary C57BL/6 (H-2b) anti-Balb/c (H-2d) MLR were set up by combining 25×10⁶ C57BL/6 splenocytes and 25×10⁶ gamma-irradiated (2000 rads) Balb/c splenocytes in a upright 25 cm2 flask with 20 ml of DMEM supplemented with 10% FBS, 15 mM Hepes, 5×10-5M 2-mercaptoethanol, 25 uM gentamicin (DME complete medium). On day 6, the surviving T cells were recovered and expanded in 24 well plates; 2×105 1° MLR T cells with 5×10⁶ irradiated Balb/c splenocytes in DME complete medium supplemented with 10 units/ml human recombinant IL-2 (hrIL-2, Chiron, Emeryville, Calif.). After two passages, 8×10⁶ anti-H-2d alloreactive T cells were added to wells of a 6-well tissue culture plate containing untreated 75% confluent monolayers of CL.7 fibroblasts in 5 ml RPMI complete media supplemented with 25 u/ml hrIL-2. The resulting CD8 T cells were passed every 3-5 days at 2×10⁶/well on CL.7 monolayers as above using 50 u/ml rhIL-2, then cloned by limiting dilution and passed every 4-6 days on CL.7 monolayers as described above. All of the CD8 T cell clones were specific for H-2d alloantigen and were CD8+CD4− by flow cytometric analysis (data not shown).

‘Epithelial’ C57BL/6 anti-H2-Kbm1 and anti-IAbm12 CD8 T cell clones were derived based on modification of our previously published methodology (33) by priming female C57BL/6 mice (H-2b) with full thickness skin grafts from female B6.C-H2bm1/ByJ and B6.C-H2bm12/KhEg mice respectively. A 3×6 mm full thickness skin graft was applied to the prepared tail dorsal surface of a C57BL/6 mouse, secured with 5-0 nylon sutures at 4 points and bandaged. The grafted skin healed in about 5 days and was rejected by day 14. Mice were rested 8 weeks after graft rejection then primed splenocytes were collected. 5×10⁶ or 8×10⁶ primed splenocytes/well were added to treated 75% confluent monolayers of Bm1.11 or Bm12.4 epithelial cells in 24- or 6-well tissue culture plates containing RPMI complete medium (RPMI 1640, 10 mM Hepes, 10% characterized FBS, 5×10-5M 2-mercaptoethanol, 25 uM gentamicin) supplemented with 500 ρg/ml IL-1a, 500 ρg/ml IL-6, 1 ηg/ml IL-7, 4 ηg/ml IL-15, 250 ρg/ml TNF-α, 3 ηM IFN4-α and 3 ηM IFN-β. First passage on alloepithelial cells was the same cytokine milieu plus 10 units/ml recombinant human IL-2 (Chiron Corporation; Seattle, Wash.). Polyclonal T cell populations were subsequently cloned by limiting dilution on mitomycin C-treated Bm1.11 or Bm12.4 alloepithelial cells to derive CD8bm1 and CD8bm12-1/CD8bm12-2 respectively. The CD8bm1 (H-2 Kbm1 specific) and CD8bm12-1, CD8bm12-2 (H-2IAbm12 specific) CD8 T cell clones were maintained on Bm1.11 and Bm12.4 alloepithelial cells respectively, pre-treated with overnight with 3.6× polykine stock solution diluted to 1× (10 units/ml hIL-2, 500 ρg/ml IL-1a, 500 ρg/ml IL-6, 1 ηg/ml IL-7, 4 ηg/ml IL-15, 250 ρg/ml TNF-α, 100 unit/ml IFN4-α and 100 units/ml IFN-β) by addition of T cells the next day. The 750K T cells are added per 1.9 cm² epithelial monolayer (1.8 ml final volume); no irradiation, fixation, or mitomycin C treatment. T cells were passed every 4-6 days as per utilization/convenience. Murine recombinant cytokines were purchased from R&D Systems (Minneapolis, Minn.).

Cytokine Analysis

T cells were activated by immobilized anti-CD3 antibody (50 μl of 0.5 μg/ml 145-2C11 per well; NA/LE BD Pharmingen, San Jose, Calif.) without or with anti-CD28 antibody (50 μl of 1.0 μg/ml 37.51 per well; functional grade; Ebioscience, San Diego, Calif.). Immobilized antibody 96-well tissue culture plates (Costar) were prepared by incubating antibody in PBS overnight at 4° C. Wells were washed once with 150 μl of media prior to use. Supernatants were collected at 24 h or 48 h as indicated for specific experiments. Relative IL-2 (1A12 and 5H4; Thermo Scientific; Rockford, Ill.), IFN-γ (XMG1.2; Thermo Scientific), IL-10 (JESS-2A5 and SXC-1; BD Pharmingen), and IL-17a (TC11-18H10.1 and TC11-8H4; Biolegend, San Diego, Calif.) levels were determined by ELISA using monoclonal antibodies according to the manufacturer's protocols. Recombinant murine IL-2, IL-10 (Thermo Scientific), IFN-γ (R&D Systems; Minneapolis, Minn.), and IL-17a (Biolegend) were used as standards.

Proliferation Assays

Proliferation assays were done with anti-CD3 (145-2C11) without or with anti-CD28 (37.51). Antibodies in 50 μl of PBS/well were used to coat 96-well tissue culture plates (Costar) overnight at 4° C. Antibodies were shaken off and wells washed 1× with 150 μl of RPMI complete media prior to their use in proliferation assays.

Experimental wells were pulsed with 0.5 μCi/well 3H-thymidine (ICN, Costa Mesa, Calif.) for 10-12 hours at 24-36 or 36-48 hours of the culture cycle as indicated. Cyclosporine A (Sigma Chemical Co.; St. Louis, Mo.) dissolved in 200 proof ethanol and CH-223191 (Calbiochem; San Diego, Calif.) dissolved in dimethylsulfoxide were used at indicated concentrations. The no treatment controls in each experiment included vehicle controls for both ethanol and dimethylsulfoxide.

Cytolytic Assays

CL.7 monolayers were suspended using enzyme free dissociation buffer. 1×10⁶ cells were labelled for 1.5 hours at 37° C. in 200 μl RPMI complete medium+100 μl 51Cr (1mCi/ml, ICN, Costa Mesa, Calif.). 51Cr labelled cells were washed twice. 5000 labelled target cells were used in four hour assays. % Specific Lysis was calculated as below:

$$\% Specific Lysis = \frac{Experimental counts - Spontaneous release}{Maximal release - spontaneous release} \times 100$$

$$Maximal release was determined with 1 \% Triton X - 100$$

Human CD8 T Cell Purification and cDNA Preparation

A protocol for obtaining peripheral blood from human volunteers without and with a history of solid organ transplantation was submitted to and approved by the Indiana University Institutional Review Board. One healthy volunteer, two renal transplant patients, a liver transplant patient and a rheumatoid arthritis patient identified as a sample of convenience were recruited to participate in the study. Each subject donated 15 cc of blood drawn through a peripheral vein into EDTA blood tubes. Mononuclear fraction was purified by centrifugation as per the manufacturer's protocol (Lymphoprep; Axis-Shield, Oslo, Norway). Purified cells were adhered to tissue culture treated 100 mm petri dishes for 1 h to remove some monocyte/macs and allow cells to disaggregate.

Non-adherent cells were recovered and then the “untouched” CD8 fraction was purified by magnetic bead separation according to the manufacturer's protocol (Miltenyi Biotech; Auburn, Calif.). Purified CD8 T cell fractions were stained for flow cytometry, and total RNA isolated (RNAeasy; Qiagen, Valencia, Calif.). 2 μg of total RNA per subject was converted to cDNA per manufacturers protocol (iScript™ cDNA Synthesis Kit; Biorad, Hercules, Calif.) and stored at −80 C.

Monoclonal Antibodies and Flow Cytometry

Murine T cells were stained with antibodies specific for murine CD4 (FITC-coupled YTS191.1; Cedarlane Laboratories; Burlington, N.C.) and CD8β (PE-coupled 53-5.8; BD Biosciences). Human T cells were stained with antibodies specific for CD4 and CD8a (RPA-T4 FITC, RPA-T8 PE; Ebioscience) and analyzed on FacsCalibur Flow Cytometer.

RT-PCR for Mouse Genes

Total RNA was isolated from CD8bm1 and CD8bm12-1 using RNAeasy (Qiagen; Valencia, Calif.). Indicated quantities of total RNA were amplified with a one-step RT-PCR system (AccessQuick RT-PCR; Promega; Madison, Wis.). Primer pairs for beta actin (5′ CCTGACGGCCAGGTCATCACTATT3′, 5′ ACTCCTGCTTGCTGATCCACAT3′; pdt 358 bp), Ahr (5′ TGCTGGATAATTCATCTGGTTTTC3′, 5′ TGCCACTTTCTCCAGTCTTAATC3′; pdt 363 bp), Scin (5′ TACATGGTTTCAGATGCAAGTGG3′, 5′CTGCGGAGAACTGTGTAATTTTG3′; pdt 396 bp), Lilrb4 (5′ GGCTGAGCCAGGCTCTGTGATC3′, 5′ CCTGTCATCACCAGCTCCATGG3′; pdt 261 bp), Lcp2 (5′ GCAGGAATCACTCGCCACTGTCTC3′, 5′CTCATGGAAGGTAGTGACGGCTG3′; pdt 406 bp), Mest (5′ CCCTTGATTTCTTAGGCTTTGGC3′, 5′ CACATGTCCCACAGCTCACTC3′; pdt 406 bp), Rasgrp3 (5′CCTCACTTTTCTGGAGCATAAATC3′, 5′ AGGAGACCAACTCTGTCATTTC3′; pdt 362 bp), Prkcz (5′ AGTACGGGTTCAGCGTGGACTG3′, 5′ GTCCCAGTCTATGCTGCGGAAG3′; pdt 293 bp).

Amplifications were done with a MJ Research PTC-200 Thermal Cycler using the following program: 1) 48° C. 45 min; 95° C. 1 min, then 2) 40 cycles 95° C. 20 sec; 57° C. 20 sec; 72° C. 40 sec, then 3) 72° C. 7 min; 4° C. hold. PCR products were separated electrophoretically on 2% agarose gels containing Ethidium Bromide; inverted images are shown for presentation purposes.

Gene Expression Microarray Analysis

CD8 T cell clones CD8bm1 and CD8bm12-1 were activated in 12-well tissue culture plates (Costar) coated at 4° C. overnight with 0.75 ml anti-CD3 antibody 145-2c11 0.5 μg/ml in PBS. Wells were washed 1× with RPMI media, then 3×10⁶ T cells added to each well in the absence and presence of 1 μg/ml cyclosporin A (Sigma Chemical Company; St. Louis, Mo.). Total RNA was isolated from each T cell clone 14 hours later using a protocol that included an RNAse-free DNAse I treatment step (RNAeasy; Qiagen, Valencia, Calif.). The experiment was repeated four times to minimize background noise. With assistance from The Indiana University Center for Medical Genomics, gene expression patterns were analyzed using the Affymetrix Mouse ST 1.0 Array that analyzes 28,853 murine genes. Genes up or down regulated 5-fold with p values<0.001 were considered in the final analysis (entire data set).

RT-PCR for Human Biomarkers

Indicated quantities of cDNA were amplified with iTaq™ DNA Polymerase according to the manufacturer's protocol (Biorad). Primer pairs for beta actin (5′ CCTGACGGCCAGGTCATCACTATT3′, 5′ ACTCCTGCTTGCTGATCCACAT3′; pdt 358 bp), Ahr (5′TGCTGGATAATTCATCTGGTTTTC3′, 5′ TGCCACTTTCTCCAGTCTTAATC3′; pdt 363 bp), Scin (5′ TACATGGTTTCAGATGCAAGTGG3′, 5′ CTGCGGAGAACTGTGTAATTTTG3′; pdt 396 bp), Pla2g4a(5′ATCTCTACAACCCCTGACAGCAG3′, 5′TTCATCACACCAGAGAATCCCAC3′; pdt 482 bp), Lilrb4 (5′GGCTGAGCCAGGCTCTGTGATC3′, 5′CCTGTCATCACCAGCTCCATGG3′; pdt 261 bp), Lcp2 (5′ GTCCTTTGAAGAAGACGATTATG3′, 5′ AGGTTTCGTGCTTCTGTCTATTG3′; pdt 406 bp) were designed with Vector NTI 11.0.

Software (Life Technologies; Grand Island, N.Y.). Amplifications were done with a MJ Research PTC-200 Thermal Cycler using the following program: 1) Hot start 95° C. 3 min, then 2) 40 cycles 95° C. 20 sec; 57° C. 20 sec; 72° C. 40 sec, then 3) 72° C. 7 min; 4° C. hold. PCR products were separated electrophoretically on 2% agarose gels containing Ethidium Bromide; inverted images are presented in individual figures.

Results

As reported herein, during the process of modelling T cell interactions with reproductive tract epithelial cells using the C57BL/6 versus bm12 transplant model, CD8 T cell clones specific for I-A^{bm12} that secreted IFN-γ and a had low cytolytic ability were isolated. One of these CD8 T cell clones, CD8bm12-1, was subsequently used in activation experiments including cyclosporin A, and found to be intrinsically resistant to that drug. That I-A^{bm12}-specific T cell had never been exposed to cyclosporin A in vivo or in vitro prior to those experiments, and was maintained in culture without cyclosporin A with continued resistant to cyclosporin A for over 3+ years. This cyclosporin resistant CD8 T cell clone was derived from a C57BL/6 mouse that had rejected a bm12 skin graft. T cells from the bm12-skin-graft-primed C57BL/6 mice were cultured ex vivo in a novel nonhematopoetic system using semi-professional bm12-origin oviduct epithelial cells as allogeneic APC Like post-transplant cardiac allograft endothelial and smooth muscle cells and tracheal epithelium, the oviduct epithelial cell lines used herein express cell surface MHC class II molecules. Without intending to be restricted to, or limited by, any specific model or hypothesis, these results are consistent with a model of chronic allograft rejection in which a cyclosporin-resistant CD8 T cell subset activated by allogeneic epithelial cells (bronchiolitis obliterans syndrome), smooth muscle cells and endothelial cells (chronic allograft vasculopathy) mediates the development of fibrosis and intimal proliferation leading to organ failure. The semi-professional T cell culture system reposted herein, and the unexpected CD8 T cell clones derived using it, provide important insights into the nonhematopoetic immunobiology of solid organ transplant rejection.

Chlamydia trachomatis is an intracellular bacterium that replicates almost exclusively in a single layer of epithelial cells lining the reproductive tract (nonhematopoetic microenvironment), thereby posing a unique problem for the host cellular immune response. As reported herein, cloned murine oviduct epithelial cell lines were derived and used to investigate Chlamydia pathogenesis in the murine model for human Chlamydia trachomatis genital tract infections (31, 32, 34). Studies using cloned oviduct epithelial cell lines isolated from B6.C-H2^{bm1}/ByJ and B6.C-H2^{bm12}/KhEg mice have been published (31, 32). Both of these cell lines express low basal levels of MHC class II that can be up regulated with IFN-γ exposure. After successful cloning a C57BL/6 oviduct epithelial cell line designated C57epi.1, it is now possible to assemble a panel of congenic oviduct epithelial cell lines with the following MHC class I (K & D loci) and MHC class II (I-A locus) alleles listed in Table 1.

TABLE 1
MHC Haplotype.

| Mouse strain (cell line) | K | I-A | D |
|---|---|---|---|
| C57BL/6 (C57epi.1) | b | b | b |
| B6.C-H2^{bm1}/ByJ (Bm1.11) | bm1 | b | b |
| B6.C-H2^{bm12}/KhEg (Bm12.4) | b | bm12 | b |

The Bm1.11 and Bm12.4 cell lines are referred to herein as Bm1epi and Bm12epi respectively. These epithelial cell lines were derived, at least in part, to study Chlamydia-specific T cells from the reproductive tracts of Chlamydia-immune mice, and to map MHC restriction elements of Chlamydia-specific T cell clones. Before attempting to isolate Chlamydia-specific T cell lines on infected oviduct epithelial APC, an alloantigen was used to model T cell-epithelial cell interactions.

Briefly, oviduct epithelial cell lines were derived from C57BL/6, B6.C-H2^{bm1}/ByJ and B6.C-H2^{bm12}/KhEg mice to take advantage of the pre-existing transplant rejection model for studying CD4 (bm12 model) versus CD8 (bm1 model) T cell biology. The B6.C-H2^{bm1}/ByJ mouse strain (bm1) is isogenic with the C57BL/6 strain except for a 3 amino acid change in the MHC class I K^{b} allele (35). This small change in the MHC class I K^{b} gene is recognized as foreign by C57BL/6 CD8 T cells, and therefore C57BL/6 mice reject bm1 tissue via an MHC class I-CD8 T cell-mediated mechanism (36, 37). The B6.C-H2^{bm12}/KhEg strain is isogenic with the C57BL/6 strain except for a 3 amino acid change in the MHC class II I-A^{b} beta chain gene (38). This small change in the MHC class II β chain is recognized as foreign by C57BL/6 CD4 T cells, and therefore (at least historically) C57BL/6 mice reject bm12 tissue via a CD4 T cell-mediated mechanism (36, 37). The biologically intact epithelial cell lines disclosed herein have IFN-γ inducible expression of MHC class II (31, 32). MHC class II up regulation on epithelial and endothelial cells is a histopathologic feature of allo-organ rejection (30, 39, 40).

Accordingly, one use for this newly isolated oviduct epithelial cell line panel is to examine conventional CD8 T cell-epithelial interactions using the Bm1epi cell line, and unconventional CD4 T cell-epithelial interactions using the Bm12epi cell line. Because the function of epithelial cells as APC is controversial (41), the “alloantigen first” approach reported herein provided the opportunity to develop at least in vitro protocols for subsequent isolation of Chlamydia-specific CD8 and CD4 T cells on infected epithelial antigen presenting cells.

Using a variant of our published protocol (33) and as described herein, T cell lines and clones were derived from C57BL/6 mice primed with full thickness allogeneic skin grafts using the epithelial cell lines as APC in vitro. Skin, though not vascular in origin, is composed of stroma including blood vessels with an overlying specialized epithelium. Memory T cells from skin-graft-primed mice recovered using allogeneic oviduct epithelial APC in vitro include, by definition, T cell subsets able to interact with epithelial cells without becoming anergized. T cells recovered using this experimental approach provided a powerful insight into T cell interactions with semi-professional antigen presenting cells such as endothelial and epithelial cells. This experimental model provides an insight into T cell immunobiology operative in the semi-professional microenvironments of solid organs and vascular walls during development of chronic allograft rejection.

For isolation of bm1-specific CD8 T cell clones, C57BL/6 female mice were tail grafted with B6.C-H2^{bm1}/ByJ full thickness skin grafts that were rejected by day 14 post-transplantation. The mice were rested for eight additional weeks before harvesting splenocytes (memory T cells) and plating them on uninfected monolayers of Bm1epi. To isolate bm12-specific CD4 T cell clones, C57BL/6 female mice were tail grafted with B6.C-H2^{bm12}/KhEg full thickness skin grafts; splenocytes were harvested as above and plated on uninfected monolayers of Bm12epi. To mimic the cytokine milieu at the epithelial interface during genital tract infections, the lymphocyte culture medium included recombinant IL-1, IL-6, IL-7, IL-15, TNF-α, IFN-α and IFN-β at concentrations reflecting levels made by oviduct epithelial cells when infected by C. muridarum (32). IL-1, IL-6, IL-7 and TNF-α are all found in human cardiac allografts undergoing rejection (42). Though IFN-α/β mRNA has not been documented in allograft rejection, human vascular smooth muscle cells treated with TNF-α up regulate IFN-β mRNA (43), and rejecting cardiac allografts have significant IFN-γ (44) which has overlapping biological activities with IFNα/β. IL-15 is constitutively made by stromal cell types and its expression is up regulated in arterial wall injury (45). The culture medium also contained a small amount of IL-2. The T cells recovered in these experimental systems grew out in a cytokine milieu similar to that documented in rejecting cardiac allografts.

Memory T cells recovered from mice primed with bm1 skin grafts vigorously responded to the Bm1epi oviduct epithelial cells in culture. A MHC class I-restricted K^{bm1}-specific CD8 T cell clone designated CD8bm1 was derived by limiting dilution and kept for further studies. CD8bm1 recognizes bm1 bone marrow-derived macrophage cell populations (BMDM), but not syngeneic C57BL/6 BMDM, or C57BL/6 BMDM pulsed/cross-primed with Bm1epi cell membranes (data not shown). These results are consistent with ‘direct’ allorecognition of the K^{bm1} molecule. CD8bm1 makes IFN-γ, and on that basis it is a conventional alloreactive “Tc 1” CD8 T cell clone that recognizes a classic MHC class I alloantigen, K^{bm1}.

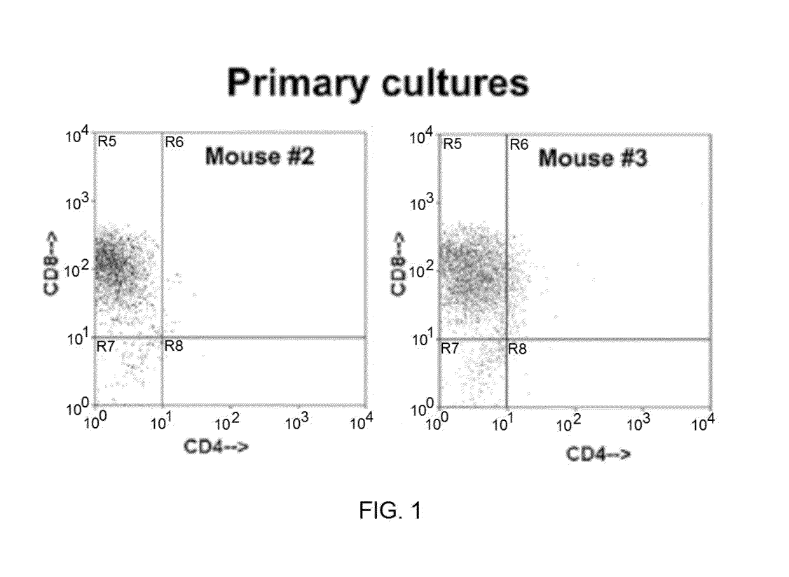

In part, because MHC class II restricted antigen presentation by epithelial cells is both controversial and unexpected, experiments were conducted to determine whether alloreactive CD4+ T cells could be derived on the Bm12epi cell line. Briefly, splenic memory T cells harvested from C57BL/6 mice primed with bm12 skin grafts responded vigorously when plated on the Bm12epi. All wells in this assay had greater than 1000 ρg/ml IFN-γ, epithelial monolayers were lysed, and activated T cells readily recovered on day 5 of the primary cultures. The same bm12-primed C57BL/6 splenocytes did not respond to Bm1epi (H-2K^{bm1}). Additionally, unprimed naïve C57BL/6 mouse splenocytes co-cultured with Bm12epi were not significantly activated, and T cells could not be recovered on day 5 of those primary cultures. IFN-γ production, lysis of the epithelial monolayers and recovery of T cells on culture day 5 were dependent on previous priming with a bm12 allogeneic skin graft. This pattern indicates that the bm12-specific T cell lines recovered in this epithelial-APC-based system originated from a population of T cells expanded during rejection of the bm12 skin grafts. One of the surprising results of the study is that all the bm12-primed/Bm12epi-derived T cell lines, 3 of 3 from three different mice were >99.5% CD8⁺ T cells rather than the expected CD4⁺ T cells (mouse 2 and mouse 3 fifth passage polyclone lines shown in FIG. 1). Two CD8⁺ I-A^{bm12}-specific T cell clones, designated CD8bm12-1 and CD8bm12-2, derived from two different mice were kept for further study. At the same time, consistent with previously published data (36), standard C57BL/6 anti-irradiated bm12 splenocyte mixed lymphocyte reactions (MLR) yielded predominantly CD4⁺ T cells with CD4 to CD8 ratios of ˜10 to 1 (data not shown), suggesting that the immunobiology of solid organ transplant rejection differs significantly from that of standard mixed lymphocyte reactions.

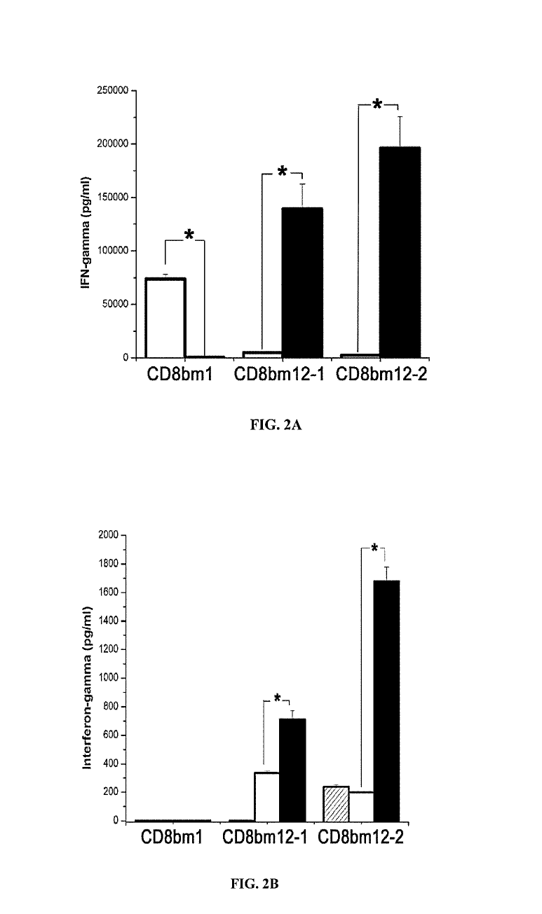

The antigen specificity of the epithelial-derived CD8 T cell clones was measured by activating them with Bm12epi (I-A^{bm12}) and Bm1epi (K^{bm1}) epithelial cells. As illustrated in FIG. 2A, the CD8bm1 T cell clone recognized Bm1epi, but not Bm12epi, which is consistent with recognition of the MHC class I allo-K^{bm1} molecule. Conversely, the CD8bm12-1 and CD8bm12-2 T cell clones recognized Bm12epi, but not Bm1epi, consistent with specificity for the MHC class II allo-1-A^{bm12} molecule. The MHC class II specificity of the CD8bm12-1 and CD8bm12-2 T cell clones was further tested by transfecting CL.7 fibroblasts (MHC class II negative, Balb/c H-2^{d} origin) with invariant chain (ATCC# MGC-6517), I-A^{b} alpha chain (ATCC# MGC-30249) and I-A^{bm12} beta chain (cloned and sequenced from Bm12epi), and subsequently isolating a cloned CL.7 cell line expressing cell surface MHC class II I-A^{bm12} after flow cytometry sorting (data not shown) designated CL.7I-A^{bm12}. The CD8bm12-1 and CD8bm12-2 T cell clones, but not the CD8bm1 T cell clone, recognized the CL.7I-A^{bm12} cell line (FIG. 2B). Recognition of I-A^{bm12} in the setting of the non-self MHC class I molecules K^{d} and D^{d} essentially ruled out endogenous cross-priming within the Bm12epi cell line; i.e. CD8bm12-1 and CD8bm12-2 do not recognize I-A^{bm12} peptide fragments loaded onto K^{b} or D^{b} MHC class I molecules. Furthermore, the CD8bm12-1 and CD8bm12-2 T cell clones were not activated by syngeneic C57BL/6 BMDM cross-primed with Bm12epi cell membranes, nor were these clones activated by syngeneic BMDM pulsed with three overlapping 20-mer peptides containing the 3 amino acid change in I-A^{b} that constitutes the I-A^{bm12} allele (data not shown).

The I-A^{bm12} beta chain allele was examined to determine if it had any of the predicted CD8 T cell epitopes on MHC class I K^{b}, D^{b}, K^{d} and D^{d} molecules utilizing the MAPPP prediction algorithm. No bm12-specific peptide fragments were predicted to bind to K^{b}, D^{b}, or D^{d}. The MAPPP algorithm predicted a single I-A^{bm12} specific-peptide (EYWNSQPEFL, SEQ ID No. 1), containing one of the three amino acids (F) that differ between C57BL/6 and bm12 mice, that may bind K^{d}. CL.7 fibroblasts pulsed with EYWNSQPEFL, SEQ ID No. 1 were not recognized by either CD8bm12-1 or CD8bm12-2 (data not shown).

CD8bm1 is a conventional MHC class I-restricted alloreactive CD8 T cell that ‘directly recognizes’ H-2K^{bm1}. CD8bm12-1 and CD8bm12-2 are MHC class II-specific alloreactive CD8 T cells that ‘directly recognize’ the MHC class II I-A^{bm12} molecule. MHC class II-specific CD8 T cells have been isolated from standard MLRs (46, 47), including C57BL/6 versus bm12 (48), virus-infected animals (49, 50) and CD4-deficient transgenic mice expressing a MHC class II-specific TCR transgene (51). Because CD8 T cells were a small minority in standard C57BL/6 versus bm12 mixed lymphocyte reactions (<10%), but the dominant population in these nonhematopoetic allo-system (>99.5%), an explanation consistent with these results is that MHC class II-restricted CD8 T cells may be more common in nonhematopoetic microenvironments of solid organs and the vascular walls. Also, consistent with these results is that the CD8 T cells reported herein recognize MHC class II I-A^{bm12} and contribute to cardiac allograft vasculopathy as demonstrated by Fischbein et al. in the C57BL/6 versus bm12 cardiac allograft model (13, 18, 19) ‘directly’ rather than ‘indirectly.’

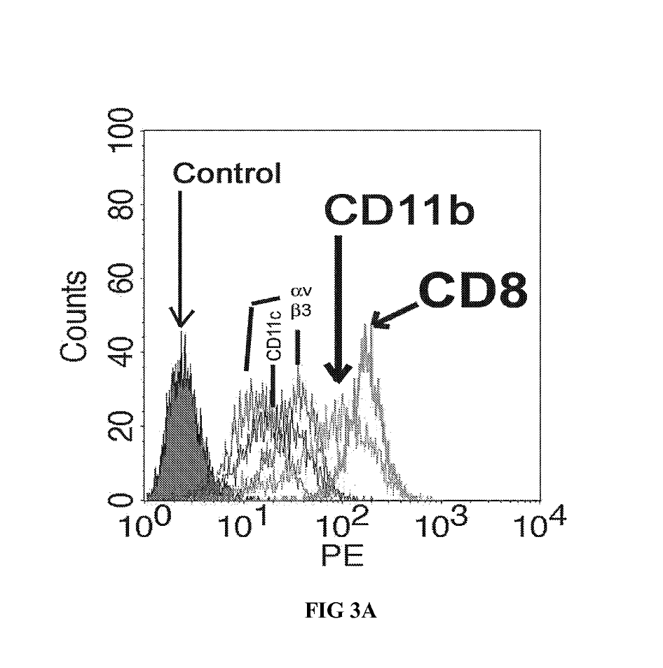

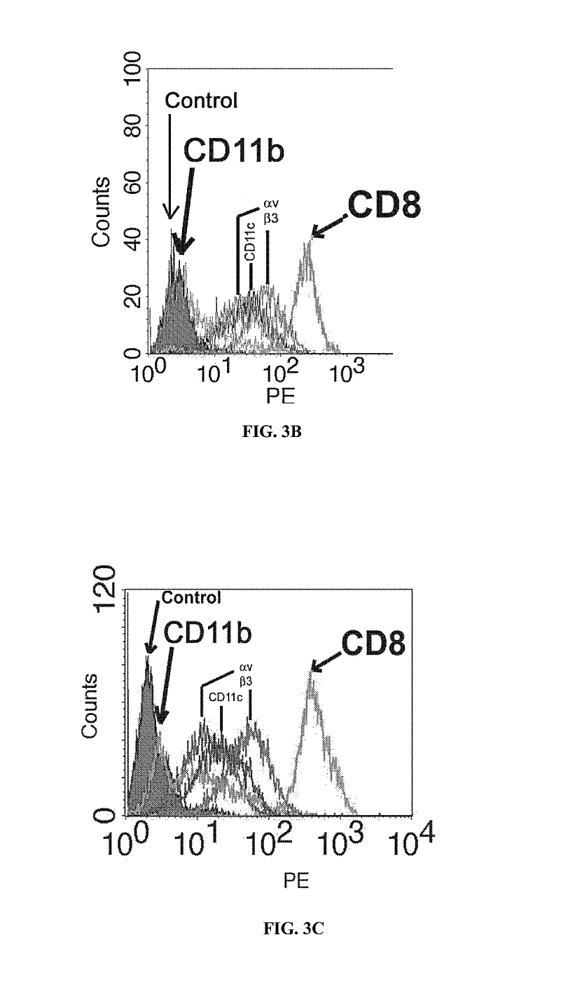

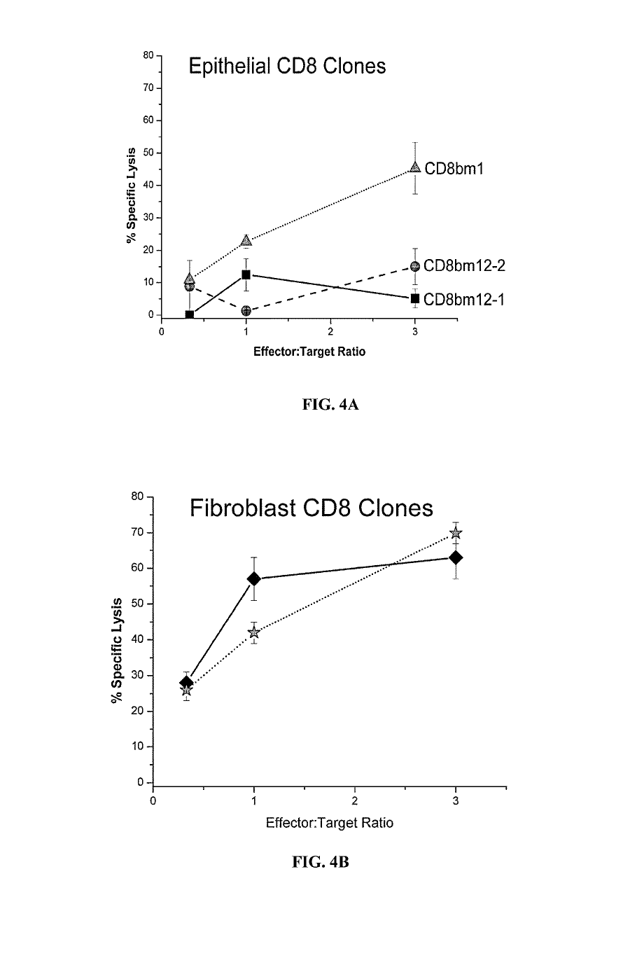

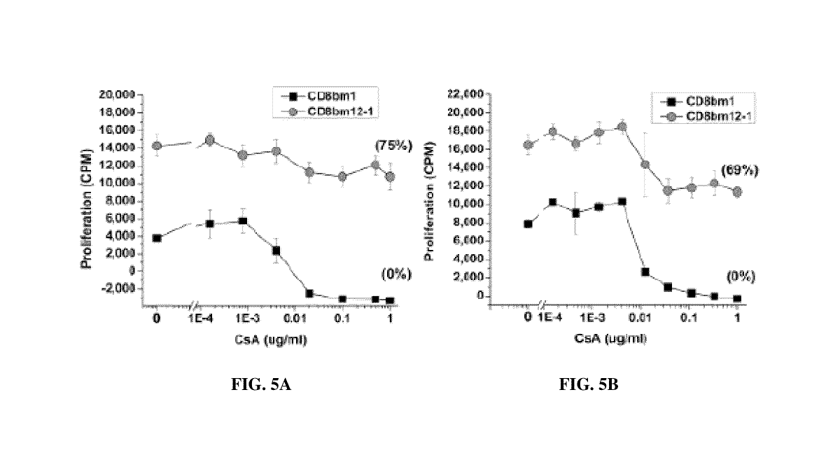

Referring now to FIGS. 3A-3C, it is shown that the MHC class I-restricted CD8bm1 (FIG. 3A) and MHC class II-restricted CD8bm12-1 (FIG. 3B) and CD8bm12-2 (FIG. 3C) T cell clones are CD8+. Following this determination, it was tested as to whether these MHC class II-specific CD8 T cells could function as cytolytic T lymphocytes. Specifically, referring now to FIGS. 4A and 4B, the cytolytic function of CD8 T cell clones was measured in four hour lactate dehydrogenase and chromium release assays. The data presented in FIG. 4A were determined with the epithelial-derived CD8 T cell clones CD8bm1, CD8bm12-1 and CDbm12-2 measured against their respective alloepithelial cell line targets Bm1epi and Bm12epi. The data presented in FIG. 4B were collected with two fibroblast-derived alloreactive CD8 T cell clones specific for H-2^{d} measured against CL.7 fibroblast targets. The CD8bm1 T cell clone cytolysis of Bm1epi cells measured at an effector to target ratio of 3:1 is similar to that of the fibroblast-derived T cell clones, while the CD8bm12-1 and CD8bm12-2 clones do not demonstrate meaningful cytolysis of Bm12epi in short term CTL assays. Both of the MHC class II-specific CD8 T cell clones CD8bm12-1 and CD8bm12-2 were significantly less cytolytic than the conventional CD8bm1 and the “fibroblast-derived” CD8 T cell clones, a phenotype consistent with cyclosporin-resistant noncytolytic CD8 T cells mediating CAV in mice treated with cyclosporin A (12, 14).

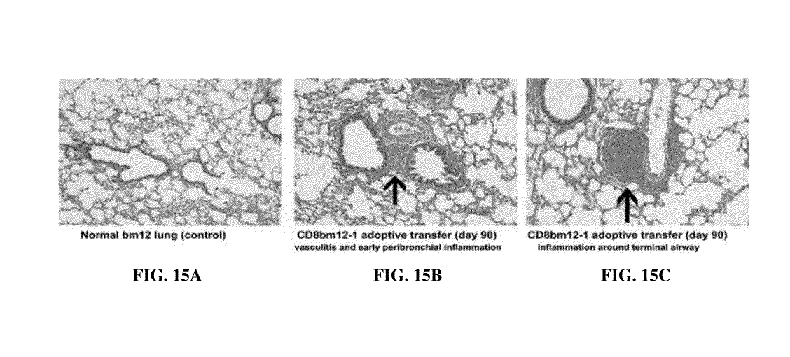

As previously referenced, bronchiolitis obliterans syndrome (BOS) is a form of chronic lung allograft dysfunction in which the bronchioles are compressed and narrowed by fibrosis and/or inflammation. The histopathology of BOS is consistent with inhibitor-resistant, noncytolytic CD8 T cells driving such chronic allograft rejection. Referring now to FIGS. 15A-15C, it is shown that adoptive transfer of CD8bm12-1 causes BOS in bm12 mice. Specifically, a mouse experiment was conducted as follows: 4 C57BL/6 (control) and 4 bm12 mice were mock-transferred, and 6 C57BL/6 (control) and 6 bm12 (experimental) mice were adoptively transferred with 500 k CD8bm12-1 T cells via the tail vein and 1.5 million CD8bm12-1 T cells subcutaneously between the shoulder blades for each mouse. Mock-transferred mice were injected with saline solution only. The control C57BL/6 mice did not express the bm12 alloantigen recognized by CD8bm12-1 T cells. The animals were weighed twice a week for a month and observed over an additional 2 month time period.

No difference in weight was identified and both the C57BL/6 controls (no alloantigen) and bm12 mice (have alloantigen=I-A^{bm12}) looked normal 3 months into the experiment. One mock-transferred bm12 mouse and one bm12 mouse treated with CD8bm12-1 T cells (by tail vein injection and subcutaneously) were randomly selected and evaluated by histopathology. FIG. 15A shows a representative micrograph of stained lung sections from the mock-transferred bm12 mouse consistent with the normal histology of lung tissue. However, as seen in FIGS. 15B and 15C, the bm12 mouse treated with the CD8bm12-1 T cells showed evidence of early BOS (i.e. rejection in the lung). As such, these findings support that adoptive transfer of CD8bm12-1 T cells causes BOS in bm12 mice.

Referring now to FIGS. 5A and 5B, further implicating the CD8bm12-1/CD8bm12-2 CD8 T cell type in vasculopathy immunobiology is that CD8bm12-1 cell proliferation to an immobilized anti-CD3 stimulus (T cell receptor complex) is resistant to cyclosporin A, while the conventional CD8bm1 clones proliferation is completely inhibited by cyclosporin A under the same conditions. Specifically, CD8bm1 proliferation was completely inhibited by CsA while CD8bm12-1 was CsA-resistant, retaining between about 70-75% of its proliferation in the presence of 1 μg/m CsA despite the complete inhibition of IL-2 production at that concentration. Residual proliferation to the anti-CD3 signal in the presence of 1 μg/m CsA is shown in parentheses for each T cell clone in each Experiment as compared to the condition without CsA. As supported by the data, the CD8bm12-1 clone proliferated as well in the presence of 1 ug/ml of cyclosporin as the CD8bm1 cell did in the absence of the inhibitor. The lower level of proliferation of CD8bm12-1 in the presence of cyclosporin is consistent with the chronicity of chronic allograft rejection (years rather than weeks).

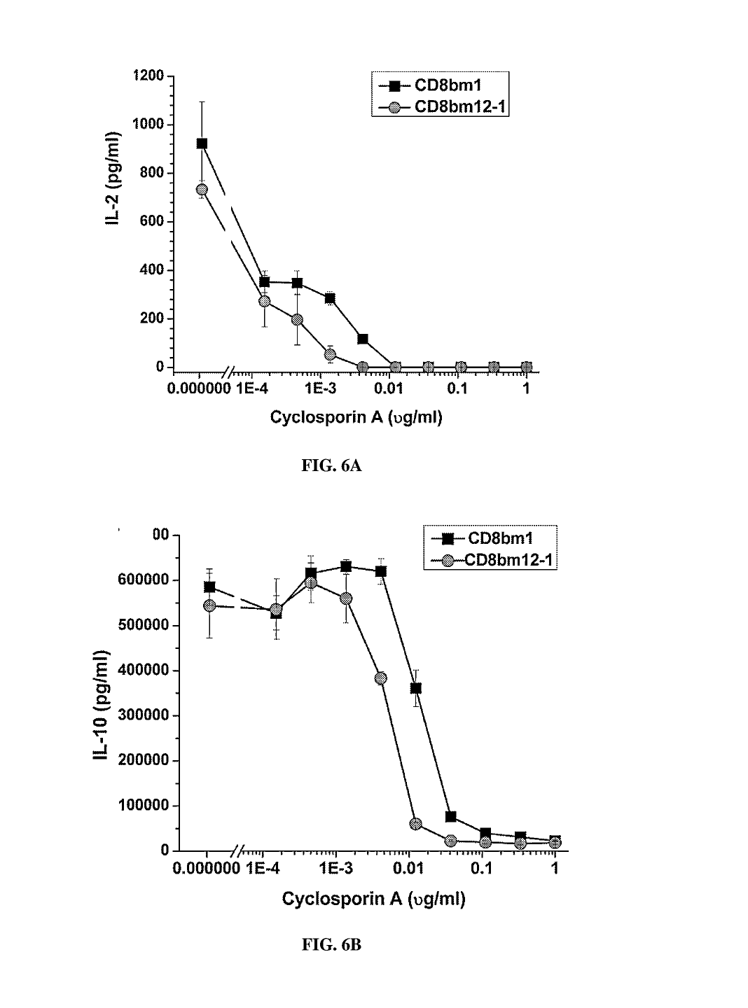

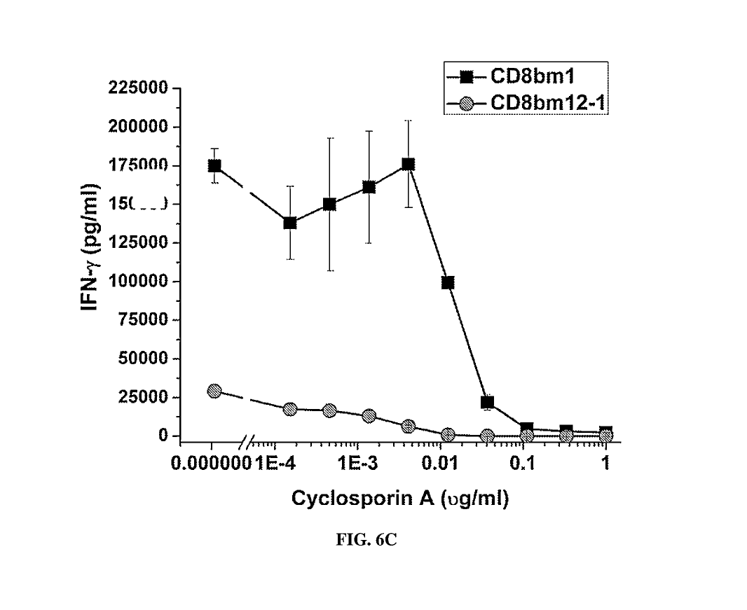

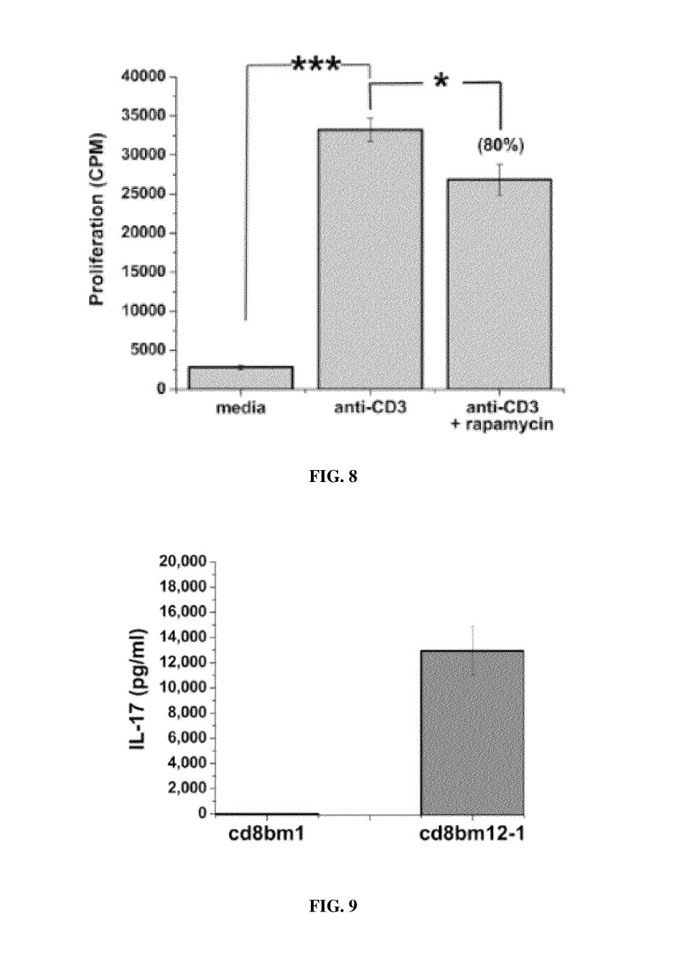

The residual proliferation shown in FIGS. 5A and 5B, occurring in the presence of sufficient CsA to completely block IL-2 production (FIG. 6A), suggests that CD8bm12-1 is also intrinsically resistant to rapamycin that acts downstream of the IL-2 receptor. This was subsequently confirmed by performing a similar study with rapamycin. Specifically, as shown in FIG. 8, at the high end of the human therapeutic range for rapamycin, 25 nM, CD8bm12-1 retained CD8bm12-1 retained about 80% of its proliferation as compared to CD8bm12-1 T cell clone proliferation in the presence of an anti-CD3 stimulus. ³H thymidine was added at 38-48 hours to quantify proliferation.

In addition to the foregoing studies, the cytokine profiles of the CD8bm12-1 T cell clones were also investigated. Referring back to FIGS. 6A-6C, it was determined that CD8bm12-1 is very sensitive to cyclosporin inhibition of IL-2 production (FIG. 6A), IL-10 production (FIG. 6B), and IFN-γproduction (FIG. 6C). Accordingly, it is thought that the CD8bm12-1 and CD8bm12-2 CD8 clones are representative of the I-Abm/12-specific CD8 T cells causing CAV in the C57BL/6 mice grafted with the bm12 cardiac allografts. Such clones are not likely to be anomalous T cell clones isolated by chance (e.g., a spontaneous mutation in calcineurin that blocks binding of cyclosporine) due to the collected data and, specifically, the inhibitory effect of CsA on cytokine profiles. As the known mouse and human T cell activation pathways are virtually identical gene for gene, and chronic allograft vasculopathy in mice treated with cyclosporin is histopathologically virtually identical to the clinical disease in humans, the results obtained in the murine experimental model is directly applicable to humans.

Gene expression profiles were also generated using the Affymetrix microarray technology to identify genes uniquely up regulated by CsA in activated CD8bm12-1 cells. Microarrays are known in the art and consist of a surface to which probes that correspond in sequence to gene products (e.g., mRNAs, etc.) can be specifically hybridized or bound to a known position, thus leaving its fluorescent tag. Hybridization intensity data detected are acquired and processed to determine which genes are active.

Here, microarray analysis was performed comparing the novel CD8bm12-1 T cell clones to the conventional “Tc 1” CD8bm1 T cell clone using Affymetrix GeneChip® Mouse GENE 1.0 ST obtained from Affymetrix, Inc. (Santa Clara, Calif.). While Affymetrix microarray technology was utilized in this instance, it will be appreciated that microarray technology from other manufacturers and/or alternative methods for measuring gene expression profiles are known in the art and could have been employed to achieve comparable data.

The CD8bm1 and CD8bm12-1 T cell clones were activated by immobilized anti-CD3 antibody (an isolated TCR signal) in the absence and presence of 1 μg/ml cyclosporin A. This activation methodology limits the activation signal to the TCR and downstream TCR signalling pathways as no accessory molecules are engaged under these conditions. Four repetitions were performed in order to minimize background noise. Specifically, 5×10⁴ CD8bm1 and Cd8bm12-1 T cells were added to wells with immobilized anti-CD3 antibody. Total RNA was isolated at 14 hours for the microarray assay from both CD8bm-1 and CD8bm12-1 T cells, converted to cDNA through methods known in the art, and stored at −80° C. until used. Microarray determined gene expression patterns were then confirmed with independent experiment RT-PCR reactions separated on 2% agarose gels containing EtBR. Specifically, β-actin amplification was included as a control for RNA/cDNA quantity and quality (250 ηg total RNA amplified for β-actin; 500 ηg total RNA amplified for the other genes).

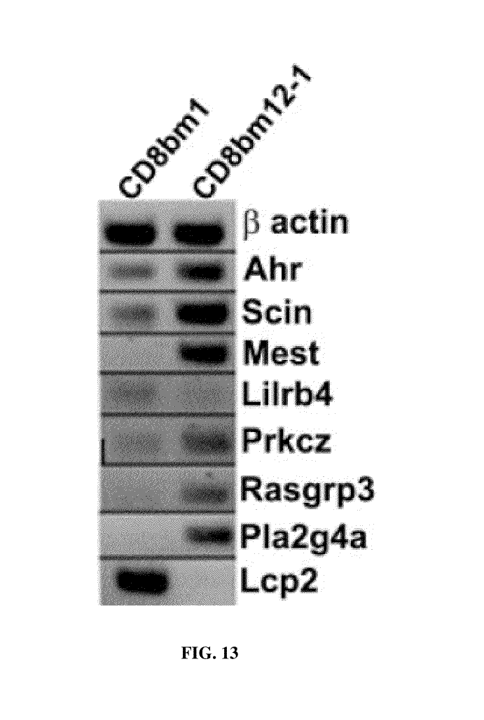

The confirmatory RT-PCR of the microarray data in FIG. 13 indicate that the CD8bm12-1 T cell clone disclosed herein represents a novel T cell lineage (see microarray Tables 2 & 3 below). Its unique features include an alternative T cell receptor signalling pathway. For example, CD8bm12-1 has several components of the B cell receptor signalling pathway including critical components PKCz_{ι} (52) and PLCγ2 (53), while also lacking or having low levels of Lcp2 and Grap2 that are critical components of the TCR signalling pathway (54) in conventional T cells. It is likely that the novel TCR signalling pathways in CD8bm12-1 contribute to its ability to proliferate in the presence of cyclosporin A, with Ca⁺⁺-independent PKCz_{ι} as a logical candidate for CsA-resistant TCR signalling. Furthermore, PKCz_{ι} has roles in activation of NF-κB, Erk and NFAT, and PLCγ2 can plausibly provide an Erk signal (52, 55). In addition, exposure of CD8bm12-1 to cyclosporin A up regulates genes that are not induced in the conventional CD8 T cell clone CD8bm1.

Table 2 shows a panel of 8 genes uniquely up regulated by cyclosporin A in anti-CD3 activated CD8bm12-1 cells as compared to identically treated CD8bm1 cells, as well as fold induction in CD8bm12-1 cells by CsA (column C2). Particularly relevant to cyclosporin resistance are the up regulation of additional B cell receptor signalling pathway components including Klh16 (56) and Rasgrp3 (55), and the aryl hydrocarbon receptor. The aryl hydrocarbon receptor (AHR) is associated with the Th17 CD4 T cell subset (57). Based on the microarray results, CD8bm12-1 appears to have a biology dependent on the AHR plus an endogenous AHR ligand, as evidenced by the 196-fold up regulation of Scin, a gene known to be up regulated by AHR activation (58) (Table 3

AHR signalling includes interaction with RelB. RelB/AHR complexes bind RelB/p52 promoters for the non-canonical NF-kB pathway (59). Recent data identifying an AHR-RelB signalling pathway (59-61) potentially tie AHR activation to T cell proliferation and survival. To investigate this interaction in light of the novel CD8bm12-1 T cells disclosed herein, a subset of mRNA biomarkers identified for CD8bm12-1 were tested and confirmed by conventional RT-PCR using the same experimental conditions as in the gene expression microarray methodology discussed herein. All mRNA markers tested showed a pattern consistent with the gene expression microarray results (see FIG. 13). Experiments in the murine CAV model suggest that with perturbation, i.e. knocking out tbet, T cell mediators of acute rejection were CD8 T cells secreting IL-17 (62). The microarray data disclosed herein shows that, at the level of mRNA that activated CD8bm12-1 differentially expressed IL-17 as compared to CD8bm1, and that IL-17 expression in activated CD8bm12-1 was blocked by CsA. Upon further investigation, this microarray finding was confirmed by activating CD8bm1 and CD8bm12-1 with anti-CD3 and measuring the levels of IL-17 present in culture supernatants (see FIG. 9). CD8bm12-1 uniquely made IL-17 to an anti-CD3 stimulus providing an mRNA independent confirmation of the microarray results. CD8bm12-1 IL-17 production was blocked by CsA, (Table 3).

Tc17 cells are a unique subset of CD8 T cells that are characterized by the secretion of IL-17. While at first blush it appears that because activated CD8bm12-1 cells have IL-17a & IL-17f mRNA they could be labelled as “Tc17” cells, they also express mRNA for TGF-β3 and make IFN-γ, which is inconsistent with assigning a Tc17 lineage. In published studies of Tc17 T cells, such cells are derived using artificial conditions including Tbet knockout mice (63) and in vitro cytokine manipulation (64). In Tbet knockout mice, the levels of IL-17 mRNA correlate with levels of RORγT, a lineage marker for Th17 T cells that suggests Tbet knockout Tc17 cells may actually be Th1 cells artifactually expressing CD8 as a consequence of lacking the critical Th1 transcription factor Tbet. In the cytokine manipulation Tc17 study, cytokine manipulation generated Tc17 T cells that reverted to Tc1 cells in vivo after adoptive transfer. In so far as it can be determine from the existing literature, no one has isolated Tc17 cells from a natural immune response, either to infectious agents, nominal antigens, or rejection of alloantigen.

The CD8bm12-1 T cell clone was isolated from wild type C57BL/6 mice that had rejected a bm12 skin graft. CD8bm12-1 is not related to the Tbet knockout mouse Tc17 subset as the microarray did not show significant mRNA for the RORγt, the transcription factor that defines the Th17 lineage and that correlates with IL-17 production by the Tbet knockout mouse Tc17 subset. Accordingly, CD8bm12-1 is not likely related to Tc17 generated in vitro by cytokine manipulation as those cells also were shown to be RORγT positive, and IL-18R negative. Indeed, the microarray confirmed that the anti-CD3 activated CD8bm12-1 is IL-18 receptor positive (mean log signal value=8.56; 29.8 fold>than conventional comparator clone CD8bm1) and does not show significant mRNA for RORγT (mean log signal value<4.5 and no different than CD8bm1). The cell surface IL-18R phenotype may be used to establish the existence of the CD8bm12-1 T cell subset as a causative agent in humans with chronic rejection, as the IL-18R can be used to sort out CD8+CD18R+ T cells from patients with chronic rejection for in vitro studies (e.g. cytokines, cyclosporin resistance, activation pathways, etc) and gene expression patterns (lineage markers, e.g. RORγT, and novel genes in Tables 2 & 3). CD7 and CD200 could potentially be similarly used as cell surface markers for sorting for the human CD8bm12-1 like CD8 T cell subset. It is possible that CD8 T cells mediating chronic rejection are uniformly CD7⁺, and that the relevant CAV CD8 T cell phenotype is CD8⁺CD7⁺CD200⁺IL-18R⁺. The CD7 finding has the additional feature in that a humanized anti-CD7 murine monoclonal antibody (SDZCHH380) has already been used in a small clinical trial to prevent acute renal graft rejection, and was shown to be safe and effective compared with anti-OKT3 (65). The humanized anti-CD7 monoclonal antibody, or its logical descendants, may be an ideal therapy for chronic allograft vasculopathy and chronic rejection of other solid organs.

TABLE 2
Cyclosporin-specific changes induced in CD8bm12-1 T cells
Genes up regulated in anti-CD3 activated CD8bm12-1 T cells by CSA:
CD8bm12-1/CSA vs CD8bm1/CSA (Condition 1 = C1)- identifies genes
unique to CSA treated CD8bm12-1 cells
CD8bm12-1/CSA vs CD8bm12-1 (Condition 2 = C2)- quantifies level
of gene induction by CSA

| Gene | C1-fold * | C2-fold † | Function |
|---|---|---|---|
| Mest | 65.8 | 10.1 | ? fxn- upregulated in adipose |
| | | | tissue expansion |
| Padi2 | 29.8 | 6.48 | Peptidyl arginine deiminase, |
| post- | | | type II- translational |
| of proteins | | | citrullination |
| Ahr | 34.95 | 2.88 | Aryl hydrocarbon receptor- |
| downstream | | | its signaling is known to be |
| | | | amplified by cyclosporin A. |
| Klhl6 | 14.35 | 3.01 | Kelch-like 6- involved in B cell |
| receptor | | | (BCR) signaling |
| Rasgrp3 | 16.4 | 5.67 | BCR signaling downstream of |
| PKCzeta | | | |
| Klhl30 | 8.89 | 9.32 | unknown- protein-protein interaction |
| domain | | | |
| Trib2 | 7.78 | 4.42 | Tribbles homolog 2- expression = |
| growth | | | a Advantage |
| ex vivo | | | |
| Rab17 | 5.5 | 5.46 | unknown- Ras family member |

* Welch T-test (log signal) pvalue <1 × 10\hat{ } − 9; Welch T-test (log signal) pvalue <1 × 10\hat{ } − 7.

Reportedly, IL-2 secretion in T cells is dependent on PKC isoform θ (66, 67). It is unlikely that IL-2 secretion by CD8bm12-1 independently explains its cyclosporin-resistant phenotype as IL-2 secretion by CD8bm1 and CD8bm12-1 was dramatically inhibited by cyclosporin A (FIG. 6A). It is reasonable to postulate that PKCζ, PLCγ2, Rasgrp3, and Klh16 participate in an alternative TCR signalling pathway that becomes especially evident in CD8bm12-1 T cells when the major IL-2-driven production/proliferation pathway through calcineurin is blocked by cyclosporin A. PKCζ is a major component of B cell receptor signalling, however, interestingly, PCKζ deficient mice also have deficiencies in T cell activation (52). The genes identified above and detailed in Tables 2 & 3 are all potentially therapeutic targets for mitigating chronic graft rejection. The AHR provides an opportunity to mitigate chronic rejection caused by the CD8bm12-1 T cell subset as the microarray shows that the AHR has been activated, based on marked up regulation of Scin, in anti-CD3 activated CD8bm12-1 T cells, with or without exposure to cyclosporin (Table 3). In Th17 cells, AHR maintains a basal level of activation, presumably by endogenous ligands, which can be manipulated to cause heighten levels of Th17 activation or Th17 suppression by synthetic ligands (57). Accordingly, it is consistent with these results is CD8bm12-1 T cells in the presence of cyclosporin A utilize a B cell-like calcineurin-independent activation pathway. B cells have a cyclosporin-resistant proliferation pathway (68). CD8bm12-1 may be the only T cell clone ever described that can proliferate in the presence of 1 μg/ml of cyclosporin A. The initial characterization provides insight into novel T cell biology that can be exploited to treat chronic allograft vasculopathy and other forms of chronic allograft rejection.

Referring now to Table 3, the differences between CD3-activated CD8bm12-1 cells and CD3-activated CD8bm1 cells independent of CsA are shown. Specifically, at least some of the important genes up and down regulated in anti-CD3 activated CD8bm12-1 vs. anti-CD3 activated CD8bm1 (Condition 3=C3), but not affected in CD3-activated CD8bm12-1 cells by addition of CSA (Condition 2=C2 same as above), are listed: i.e., gene expression that differs at baseline in the activated conventional (CD8bm1) and unconventional (CD8bm12-1) CD8 T cell clones.

TABLE 3
Baseline differences between activated CD8bm12-1 & CD8bm1 T cells.

| | | | |
|---|---|---|---|
| Gene | C3-fold‡ | C2-fold | Function |

B cell signaling

| | | | |
|---|---|---|---|
| Prkcz | 8.38 | 1.10 | PKCzeta- critical component |
| | | | of B cell receptor (BCR) |
| | | | signaling |
| PLCγ2 | 8.06 | −1.14 | Phospholipase C gamma |
| signaling | | | 2- critical component of BCR |
| Gpr183 | 7.64 | 1.46 | B cell migration |

T cell signaling

| | | | |
|---|---|---|---|
| Lcp2 | −84.47 | −2.26 | SLP-76-critical for |
| | | | conventional T cell receptor |
| | | | (TCR) signaling |
| Grap2 | −6.57 | 3.51 | LAT to SLP-76 adaptor |
| Dgkα | −16.34 | 1.14 | Diacyl glyceride kinase α- up |
| regulated in | | | anergic states |
| Lilrb4 | −18.55 | −2.27 | Leukocyte immunoglobulin- |
| receptor, | | | like subfamily B, member |
| co-inhibitory | | | 4- a receptor |

Aryl hydrocarbon receptor

| | | | |
|---|---|---|---|
| Scin | 193.18 | 1.19 | Scinderin- up regulated by |
| activation of aryl | | | hydrocarbon receptor |
| (Ahr) | | | |
| Pla2g4a | 57.32 | 1.38 | Phospholipase A2, group IV- |
| | | | signal transducer for Ahr |

Cell surface markers & cytokines

| | | | |
|---|---|---|---|
| CD7 | 28.47 | −1.12 | CD7 |
| Il8r1 | 10.91 | −2.71 | IL-18 receptor |
| CD200 | 10.38 | −5.56 | CD200 |
| IL-17a | 8.5 | −11.1 | IL-17a |
| IL-17f | 7.03 | −8.72 | IL-17f |
| Tgfb3 | 12.28 | 1.3 | TGF-beta 3 |

Other

| | | | |
|---|---|---|---|
| Sgk3 | 26.57 | −1.01 | downstream of PI-3 |
| | | | kinase (survival) |
| Gpr15 | 20.21 | 1.45 | G protein-coupled receptor 15 |
| Pls3 | 6.80 | 1.22 | unknown signaling molecule |
| Zfp187 | 9.80 | 1.03 | unknown transcription factor |

‡Welch T-test (log signal) pvalue <1 × 10\hat{ } − 7.

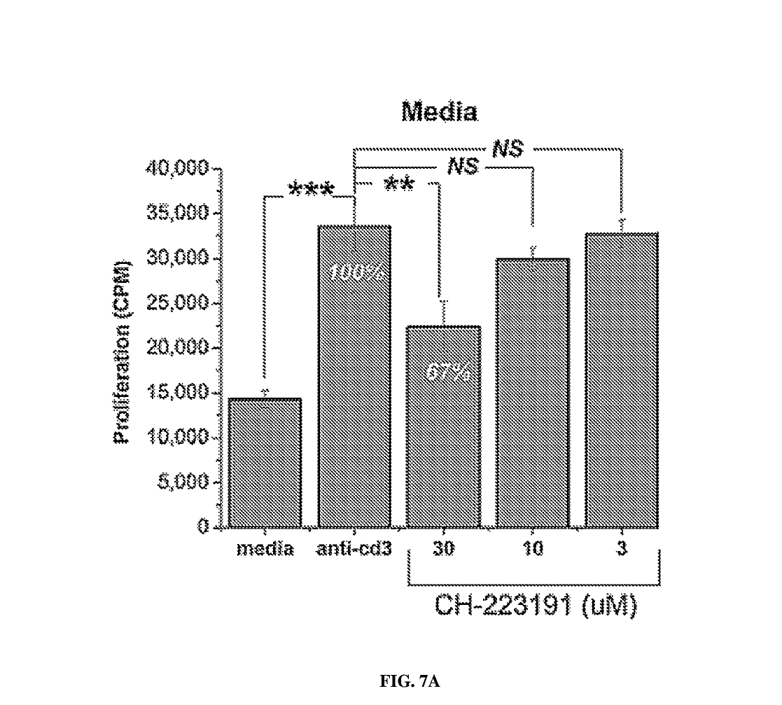

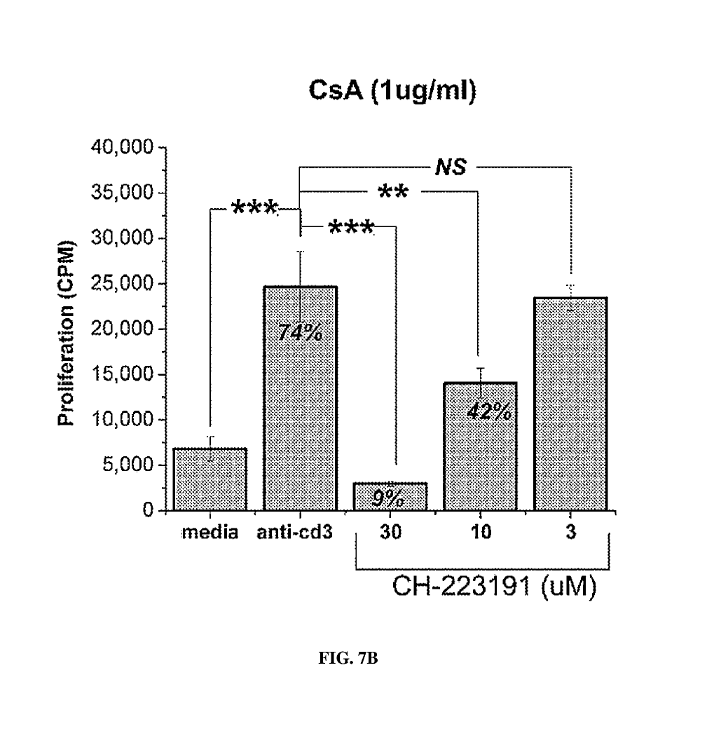

Based on the high level of Scin mRNA in CD8bm12-1 T cells proliferating in the presence of CsA as previously determined, it appears that activation of the AHR is critical to the cyclosporine-resistance pathway. To verify, experiments activating CD8bm12-1 with immobilized anti-CD3 were repeated in the presence of varying concentrations of CH-223191, a compound that antagonizes binding and activation of the aryl hydrocarbon receptor (AHR). Specifically, overall proliferation of CD8bm12-1 activated by anti-CD3 in the presence of 30 uM CH-223191 was inhibited by 33%; i.e. was only able to proliferate to about 67% the level seen when CD8bm12-1 was activated by anti-CD3 in media. FIG. 7A demonstrates that activation of the AHR is an important event in proliferation of CD8bm12-1 to an anti-CD3 (T cell receptor complex) stimulus.

Now referring to FIG. 7B, in the same experiments as shown in FIG. 7A, CD8bm12-1 was activated with immobilized anti-CD3 in media containing 1 μg/ml cyclosporin A in the presence of varying concentrations of CH-223191. In the presence of 1 μg/ml cyclosporin A, CD8bm12-1 cells retained about 74% of their overall ability to proliferate to an anti-CD3 stimulus as compared to an anti-CD3 stimulus in regular media. However, in media containing cyclosporin A, the presence of 30 μM of the hydrocarbon receptor antagonist CH-223191 reduced CD8bm1-12 proliferation to only about 9% of that to an anti-CD3 stimulus in regular media; a level of proliferation less than CD8bm12-1 in media without immobilized anti-CD3 (unactivated cells). In other words, the ability of CD8bm12-1 to proliferate to an anti-CD3 stimulus in the presence of 1 μg/ml CsA was almost completely abrogated by the addition of 30 μM CH223191.

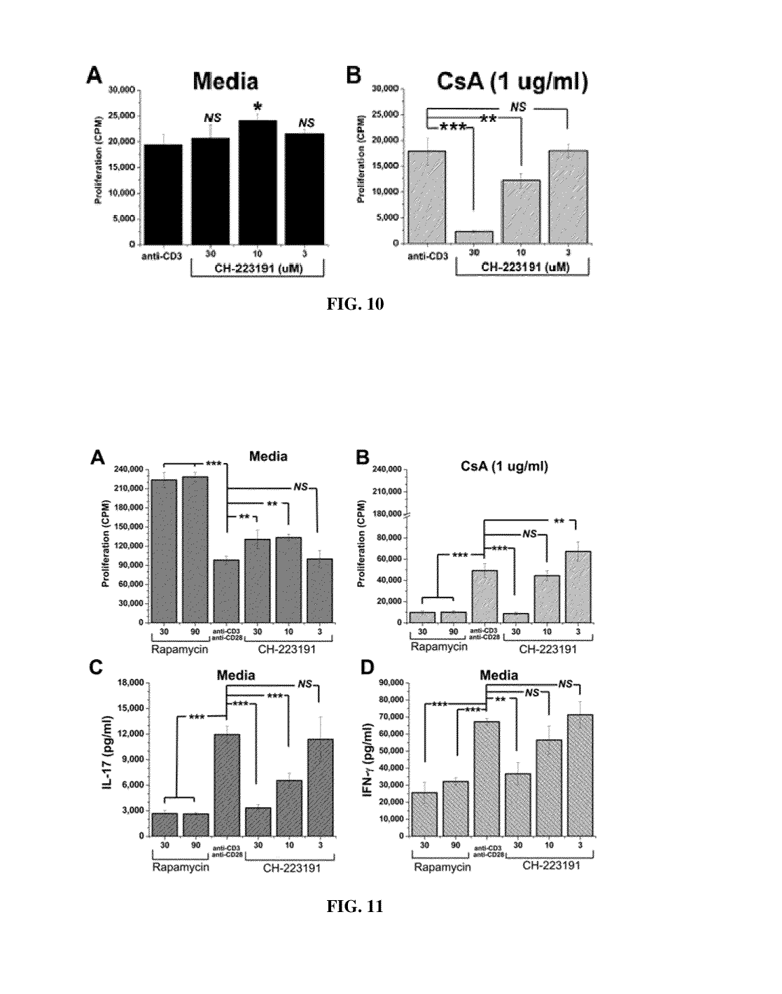

FIG. 10 shows bar graphs of data from two additional experiments performed on CD8bm12-1 T cells activated by immobilized anti-CD3 in the absence and presence of CSA and the absence and presence of varied concentrations of the AHR inhibitor CH-223191 (μM). In these graphs, proliferation in the media control (which does not contain immobilized anti-CD3 and is thus an un-activated condition) for each experimental condition was subtracted from the proliferation for the respective activated experimental condition (with anti-CD3) in order to focus on proliferation driven through TCR signalling.

The data presented in FIGS. 7A, 7B, and 10 demonstrate that aryl hydrocarbon receptor activation by an endogenous ligand is critical to the intrinsic cyclosporin resistance phenotype of CD8bm12-1, and by extrapolation, that chronic allograft rejection could be treated with cyclosporin A plus an antagonist of the AHR. In all the results presented in FIGS. 7A, 7B, and 10 are data collected from three independent experiments demonstrating that cyclosporine-resistant proliferation of the CD8bm12-1 was dependent on a functional AHR.

Referring now to FIG. 8, cell proliferation, expressed in counts of ³H thymidine, was measured for CD8bm12-1 cells exposed to media only (unactivated cells), and to media plus immobilized anti-CD-3 antibody (activated CD8bm12-1 cells) in the presence of rapamycin. Specifically, the CD8bm12-1 T cell clones were activated by immobilized anti-CD3 antibody in the presence of either 0 nM, or 25 nM rapamycin. As the data depicted in FIG. 8 illustrates and as previously discussed, rapamycin had no significant effect on proliferation of CD8bm12-1 to an anti-CD3 stimulus. Additionally, these data demonstrate that CD8bm12-1 proliferation to an anti-CD3 stimulus is largely IL-2-independent as rapamycin blocks signalling downstream of the IL-2 receptor. This conclusion is further supported by data shown in FIGS. 5A & 6A evidencing that the CD8bm12-1 T cell clone is able to proliferate to an anti-CD3 stimulus in the presence of cyclosporin A concentrations that drive IL-2 production to undetectable levels. The measurement of cell proliferation was at 24-36 hours by pulsing with ³H thymidine. Students t-test p values for the data were <0.0005. These data indicate that activated CD8bm12-1 cells are rapamycin resistant.

The known resistant mechanisms to CsA (e.g., an IL-2-dependent resistance pathway) and rapamycin are dependent on CD28 costimulation. In a conventional study, proliferation of healthy donor peripheral blood T cells stimulated via anti-CD3 alone was inhibited by CsA, but the same cells costimulated with CD3 and CD28 retained 65% of their proliferation in the presence of 1 ug/ml CsA. Further, under the costimulatory conditions, increased levels of IL-2 mRNA were detected at levels seen with untreated T cells activated by an anti-CD3 stimulus (69). A subsequent CsA study showed that IL-2 mRNA up regulation under CD28-costimulatory conditions included NFAT translocation to the nucleus (70).

While human CD4 T cells are very susceptible to rapamycin inhibition, a subset of human CD8 T cells (clones and peripheral blood) is intrinsically resistant to rapamycin. Similar to CsA resistance, rapamycin resistance in peripheral blood CD8 T cells was dependent on CD28 costimulation and IL-2 production (71). CD28 costimulatory blockade may be clinically problematic as it has been shown to be deleterious in MHC class II mismatched murine transplant model (72, 73) and, in a human clinical trial, beletacept (hCTLA4Ig) was associated with greater frequency of acute rejection episodes as compared to cyclosporine (74). There is evidence for an IL-2-independent cyclosporine-resistance pathway as a subset of murine T cells primed with alloantigen in vivo, then scored for proliferation in vitro without restimulation, were capable of proliferating in the presence of CsA and neutralizing antibody to IL-2 (75).

Accordingly, additional investigations utilizing an anti-CD3/CD28 activation signal were performed to determine whether CD8bm12-1 is resistant to CsA and rapamycin via a CD28 costimulatory mechanism (see FIG. 11). Specifically, 5×10⁴ of both CD8bm1 and CD8bm12-1 T cells were separately activated with immobilized anti-CD3/CD28 with and without 1 μg/ml CsA and in the presence and absence of varied concentrations of the AHR inhibitor CH-223191 (μM) and rapamycin (ηM). ³H thymidine was added to each group of cells at 36-48 hours to quantify proliferation, where applicable. Furthermore, where applicable, IL-17 and IFN-γ levels in culture supernatants were determined at 36 hours by ELISA for the media experimental condition; no cytokines were detectable in the presence of CsA (not shown). Aggregate data was collected from two independent experiments; *=p value<0.05, **=p value<0.005, NS=not statistically significant.

As shown in FIG. 11, the CD8bm12-1 T cells activated by an anti-CD3/CD28 signal had robust proliferative responses that were only minimally inhibited by 1 μg/ml CsA, 90 μM rapamycin, or 30 μM CH-223191. However, the combination of CsA with either CH-223191 or rapamycin proved to be a potent inhibitor of CD8bm12-1 proliferation. In other words in media containing CsA, AHR hydrocarbon inhibitor and rapamycin abrogate the CsA-resistant TCR signalling pathway in the presence of a CD28 costimulatory signal, demonstrating that the CD8bm12-1 CsA-resistance mechanism is different than that previously described in the literature.

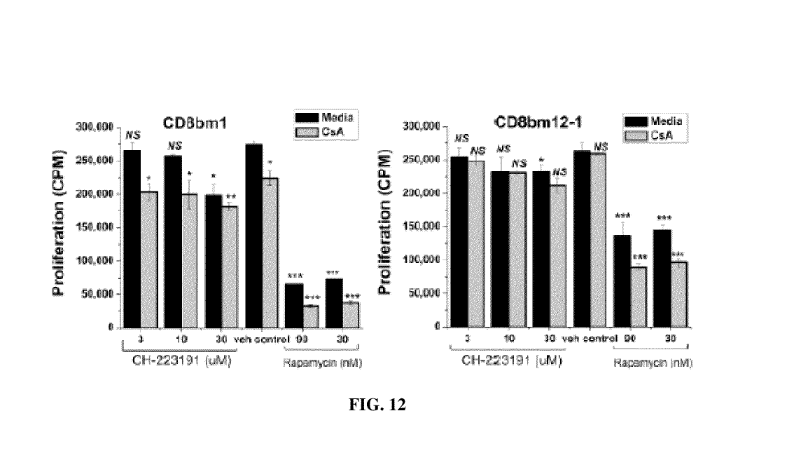

Now referring to FIG. 12, a control experiment shows that CH-223191 and CsA had minimal inhibitory effect on CD8bm1 or CD8bm12-1 homeostatic proliferation driven by IL-7 (3 ηg/ml), while rapamycin significantly inhibited IL-7 driven homeostatic proliferation in both CD8bm1 and CD8bm12-1 in the absence of an anti-CD3 signal (³H thymidine was added at 36-48 hours to score proliferation; *=p value<0.05, **, p value<0.0005, NS=not statistically significant). Further, the addition of 1 μg/ml CsA to either inhibitor had little effect on its baseline inhibition. These results suggest that rapamycin has inhibitory effects that go beyond simply blocking signalling downstream of the IL-2 receptor.

Based on these results, it was hypothesized that transplant patients with chronic rejection on calcineurin inhibitor therapies would have expansion of a cyclosporine-resistant CD8 T cell subset in their blood. For example, our novel in vitro mouse data suggests that genes associated with the AHR would be up regulated in the cyclosporine-resistant CD8 T cells circulating in the peripheral blood. However, in light of conventional teachings, this postulate is laden with negative possibilities including: 1) mouse T cells are not human T cells; 2) genes up regulated in T cells routinely stimulated for maintenance in culture will have an activated gene expression pattern not likely found in quiescent T cells circulating in peripheral blood; and 3) unless the percentage of cyclosporine-resistant T cells is significant, the signal for any mRNA biomarker will be swamped out by the majority presence of conventional CD8 T cells. This latter issue renders negative potential RNA biomarkers like Lcp2 likely unworkable.

Despite such considerations, a preliminary investigation was conducted to identify potential mRNA biomarkers for chronic rejection and rheumatoid arthritis. After receiving approval from the Indiana University School of Medicine Institutional Review Board, five subjects were recruited: 1) a healthy female without chronic medical diseases; 2) a renal transplant patient 2 years post second transplant experiencing a significant decline in function of the transplanted kidney in the 4 week interval preceding her evaluation for this study (4 ml/min decline in creatinine clearance from 41 ml/min to 37 ml/min); 3) a renal transplant patient about 6 years post-transplant with end-stage chronic rejection approaching hemodialysis with no decline in her renal function in the 6 week interval preceding her evaluation for this study (creatinine clearance stable at 19 ml/min); and 4) a liver transplant (Hepatitis C virus) patient five years post transplant. Additionally, a fifth patient presenting with rheumatoid arthritis was also recruited (a female, 79 years of age; rheumatoid arthritis present for about 12 years; cyclic citrullinated peptide Ig positive (195 units; very high)).

Both renal transplant patients were on the calcineurin inhibitor tacrolimus with therapeutic levels present in their blood. Similarly, the liver transplant patient had been on tacrolimus until 4 weeks prior to donating the blood sample. The rheumatoid arthritis patient was on leuflonamide and low dose prednisone. Each patient donated three 5 cc EDTA tubes of blood during the course of laboratory work being done as part of routine care; the healthy control volunteer donated three 5 cc ETDA tubes of blood specifically for this study. CD8 T cells were purified from each subject using sequential Lymphoprep® centrifugation and magnetic bead isolation (materials & methods).

Purified CD8 T cell populations were >88% CD8 T cells for the transplant patients; 48% for the rheumatoid arthritis patient. Total RNA was isolated from the purified CD8 T cell populations of the five subjects, converted to cDNA through methods known in the art, and stored at −80° C. until used for RT-PCR. Four positive (AHR, Prkcz, Pla2g4a and Scin) and two negative (Lcp2 and Lilrb4) RNA chronic rejection biomarkers were then screened by RT-PCR using the stored cDNA.

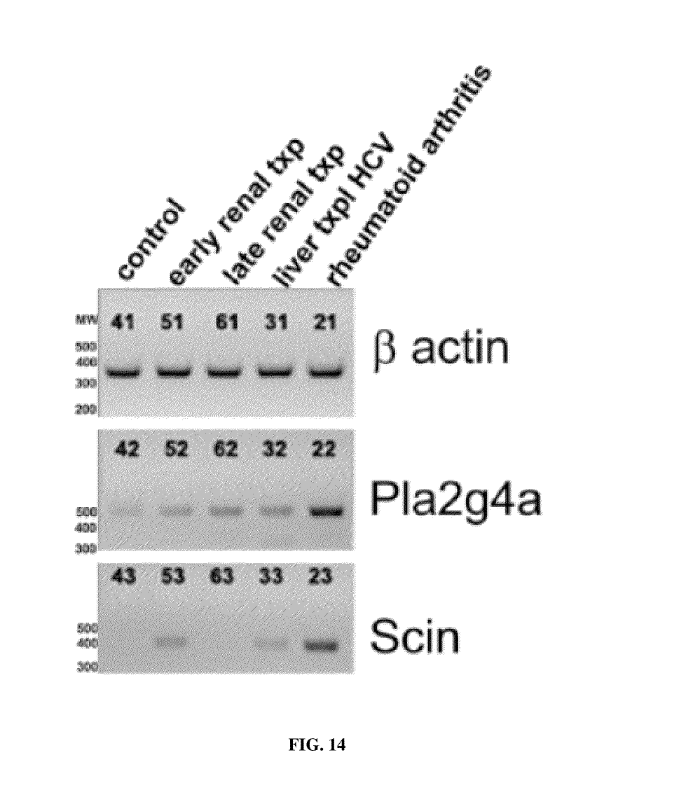

Total RNA (as converted to cDNA) in the β-actin amplification was 50 ηg equivalents; 150 ηg equivalents were amplified for Pla2g4a and Scin. There were no differences detected in levels of circulating CD8 T cell mRNA for AHR, Prkcz, Lcp2 or Lilrb4; however, there were different levels of Pla2g4a and Scin mRNA in the CD8 pool for the subjects. FIG. 14 clearly shows an increase in Pla2g4a in the three transplant patients as compared to the healthy subject, and a significant increase in Pla2g4a in the subject with rheumatoid arthritis. Similarly, Scin was elevated in the transplant patients with active rejection as defined by a drop in function of the transplanted kidney function in the 4 week interval preceding the donation and an elevation in liver transaminases, respectively. The patient with rheumatoid arthritis also had a marked elevation in Scin. Phospholipase A2 group IV (Pla2g4a) is the signal transducer for the AHR; Scinderin (Scin) is a cytoskeletal component up regulated by activation of AHR.

Accordingly, an elevated level of expression of either or both of the Scin and Pla2g4a biomarkers can be indicative of if a patient has experienced an expansion of the inhibitor-resistant, non-cytolytic CD8 T cells that drive chronic allograft rejection and/or the presentation of rheumatoid arthritis. Elevation of Scin is indicative of recent activation of the inhibitor resistant, non-cytolytic CD8 T cells that drive chronic allograft rejection and/or the presentation of rheumatoid arthritis. As such, these biomarkers can be used in diagnosing and/or monitoring such conditions. For example, in at least one embodiment, the level of expression of Scin and/or Pla2g4a may be assessed in a population of inhibitor-resistant CD8 T cells from a transplant patient to determine such patient's susceptibility to chronic allograft rejection. Because an elevated expression of either of these biomarkers correlates to an expansion of the inhibitor-resistant CD8 T cells that mediate chronic allograft rejection, the absence or existence of elevated expression of Scin and/or Pla2g4a is indicative of whether that individual is more or less susceptible to transplant rejection.

Furthermore, the a direct correlation between the expansion of inhibitor-resistant, non-cytolytic CD8 T cells and the onset of chronic allograft rejection and rheumatoid arthritis allows for such conditions to be treated by inhibiting the proliferation of such CD8 T cells. For example, in at least one embodiment, a clinician may treat rheumatoid arthritis by administering an effective dose of a compound that inhibits the proliferation of that particular subset of CD8 T cells. In at least one exemplary embodiment, the subset of inhibitor-resistant CD8 T cells expresses AHR and the compound comprises at least a calcineurin inhibitor and an AHR inhibitor; an mTOR inhibitor and an AHR inhibitor would similarly be expected to be effective based on the art in this application. The MHC class II-specific CD8 T cell clone CD8bm12-1 reported herein is intrinsically resistant to cyclosporin and rapamycin, lacks potent cytolytic activity, expresses the aryl hydrocarbon receptor and may be representative of a cyclosporin-resistant subset of CD8 T cells previously described as critical participants in murine models of cardiac allograft vasculopathy. The identifying materials and methods disclosed herein are useful for candidate ‘CAV’ and chronic allograft rejection T cell phenotypes, and activation pathways operative in the immunopathology of cardiac allograft vasculopathy and chronic allograft rejection. These materials and methods can be used to identify new therapeutic targets for treating allograft vasculopathy, and chronic T cell-mediated rejection in other solid organ transplants. Furthermore, as described herein, the materials and methods disclosed herein can be used to diagnose, monitor and treat chronic allograft rejection, rheumatoid arthritis, and any other condition associated with the elevated expression of the biomarkers identified herein.

Aspects of the invention include methods for screening, selecting and identifying molecules that can inhibit, reduce or eliminate a subset of CD8 T-cells that contribute to allograft rejections. These methods are amenable to use in both human and animals. For example, all the candidate target molecules for selectively depleting or inhibiting cyclosporin-resistant CD8 T cells (shown in Tables 2 & 3) have conserved human homologs. Representative methods for treating allograft rejection disease or for methods for selecting compounds that have the ability or potential to treat allograft rejection disease include the steps of depleting cyclosporin-resistant CD8 T cells using a pre-existing humanized anti-CD7 antibody (65), or new anti-CD7 or anti-IL-18 receptor (IL-18r1) or anti-CD200 antibody reagents developed for this or other purpose.

The cyclosporin-resistant CD8 T cell subset can predictably be purified from normal individuals, but likely these cells are more readily harvested from transplant patients on calcineurin inhibitor therapies with chronic allograft rejection by, for example, sorting on the IL-18r1⁺CD8⁺ or CD200⁺CD8⁺ T cell subset. Purification of this T cell subset can be accomplished using any means known in the art including for example, but not limited to, magnetic bead separation or cell-sorting with a flow cytometer to provide a convenient source of this novel cell type for further biochemical/molecular biology characterization, and for bioassays to identify potential inhibitors of the cyclosporin resistant proliferation pathway.

While the novel technology has been illustrated and described in detail in the figures and foregoing description, the same is to be considered as illustrative and not restrictive in character, it being understood that only the preferred embodiments have been shown and described and that all changes and modifications that come within the spirit of the novel technology are desired to be protected. As well, while the present disclosure was illustrated using specific examples, theoretical arguments, accounts, and illustrations, these illustrations and the accompanying discussion should by no means be interpreted as limiting the technology. All patents, patent applications, and references to texts, scientific treatises, publications, and the like referenced in this application are incorporated herein by reference in their entirety.

### REFERENCES

1. Lodhi, S. A., K. E. Lamb, and H. U. Meier-Kriesche. Solid Organ Allograft Survival Improvement in the United States: The Long-Term Does Not Minor the Dramatic Short-Term Success. Am J. Transplant.
2. Sundaresan, S., E. P. Trulock, T. Mohanakumar, J. D. Cooper, and G. A. Patterson. 1995. Prevalence and outcome of bronchiolitis obliterans syndrome after lung transplantation. Washington University Lung Transplant Group. Ann Thorac Surg 60: 1341-1346; discussion 1346-1347.
3. Taylor, D. O., L. B. Edwards, M. M. Boucek, E. P. Trulock, M. C. Deng, B. M. Keck, and M. I. Hertz. 2005. Registry of the International Society for Heart and Lung Transplantation: twenty-second official adult heart transplant report—2005. J Heart Lung Transplant 24: 945-955.
4. Anasetti, C., B. R. Logan, S. J. Lee, E. K. Waller, D. J. Weisdorf, J. R. Wingard, C. S. Cutler, P. Westervelt, A. Woolfrey, S. Couban, G. Ehninger, L. Johnston, R. T. Maziarz, M. A. Pulsipher, D. L. Porter, S. Mineishi, J. M. McCarty, S. P. Khan, P. Anderlini, W. I. Bensinger, S. F. Leitman, S. D. Rowley, C. Bredeson, S. L. Carter, M. M. Horowitz, D. L. Confer, Blood, and N. Marrow Transplant Clinical Trials. 2012. Peripheral-blood stem cells versus bone marrow from unrelated donors. N Engl J Med 367: 1487-1496.
5. McDyer, J. F. 2007. Human and murine obliterative bronchiolitis in transplant. Proc Am Thorac Soc 4: 37-43.
6. Panoskaltsis-Mortari, A., K. V. Tram, A. P. Price, C. H. Wendt, and B. R. Blazar. 2007. A new murine model for bronchiolitis obliterans post-bone marrow transplant. Am J Respir Crit. Care Med 176: 713-723.
7. Fernandez, F. G., A. Jaramillo, C. Chen, D. Z. Liu, T. Tung, G. A. Patterson, and T. Mohanakumar. 2004. Airway epithelium is the primary target of allograft rejection in murine obliterative airway disease. Am J Transplant 4: 319-325.
8. Higuchi, T., A. Jaramillo, Z. Kaleem, G. A. Patterson, and T. Mohanakumar. 2002. Different kinetics of obliterative airway disease development in heterotopic murine tracheal allografts induced by CD4+ and CD8+ T cells. Transplantation 74: 646-651.
9. Jungraithmayr, W., P. Vogt, I. Inci, S. Hillinger, S. Arni, S. Korom, and W. Weder. A model of chronic lung allograft rejection in the rat. Eur Respir J 35: 1354-1363.
10. Wasowska, B., K. J. Wieder, W. W. Hancock, X. X. Zheng, B. Berse, J. Binder, T. B. Strom, and J. W. Kupiec-Weglinski. 1996. Cytokine and alloantibody networks in long term cardiac allografts in rat recipients treated with rapamycin. J Immunol 156: 395-404.
11. Servais, A., V. Meas-Yedid, O. Toupance, Y. Lebranchu, A. Thierry, B. Moulin, I. Etienne, C. Presne, L. B. Hurault de, P. Le Pogamp, Y. Le Meur, D. Glotz, C. Hayem, J. C. Olivo Marin, and E. Thervet. 2009. Interstitial fibrosis quantification in renal transplant recipients randomized to continue cyclosporine or convert to sirolimus. Am J Transplant 9: 2552-2560.
12. Skaro, A. I., R. S. Liwski, J. Zhou, E. L. Vessie, T. D. Lee, and G. M. Hirsch. 2005. CD8+ T cells mediate aortic allograft vasculopathy by direct killing and an interferon-gamma-dependent indirect pathway. Cardiovasc Res 65: 283-291.
13. Schnickel, G. T., D. Whiting, G. R. Hsieh, J. J. Yun, M. P. Fischbein, M. C. Fishbein, W. Yao, A. Shfizadeh, and A. Ardehali. 2004. CD8 lymphocytes are sufficient for the development of chronic rejection. Transplantation 78: 1634-1639.
14. Vessie, E. L., G. M. Hirsch, and T. D. Lee. 2005. Aortic allograft vasculopathy is mediated by CD8(+) T cells in Cyclosporin A immunosuppressed mice. Transpl Immunol 15: 35-44.
15. Kosaka, H., C. D. Surh, and J. Sprent. 1992. Stimulation of mature unprimed CD8+ T cells by semiprofessional antigen-presenting cells in vivo. J Exp Med 176: 1291-1302.
16. Shi, C., W. S. Lee, Q. He, D. Zhang, D. L. Fletcher, Jr., J. B. Newell, and E. Haber. 1996. Immunologic basis of transplant-associated arteriosclerosis. Proc Natl Acad Sci USA 93: 4051-4056.
17. Szeto, W. Y., A. M. Krasinskas, D. Kreisel, A. S. Krupnick, S. H. Popma, and B. R. Rosengard. 2002. Depletion of recipient CD4+ but not CD8+ T lymphocytes prevents the development of cardiac allograft vasculopathy. Transplantation 73: 1116-1122.
18. Fischbein, M. P., J. Yun, H. Laks, Y. Irie, M. C. Fishbein, B. Bonavida, and A. Ardehali. 2002. Role of CD8+ lymphocytes in chronic rejection of transplanted hearts. J Thorac Cardiovasc Surg 123: 803-809.
19. Fischbein, M. P., J. Yun, H. Laks, Y. Irie, M. C. Fishbein, M. Espejo, B. Bonavida, and A. Ardehali. 2001. CD8+ lymphocytes augment chronic rejection in a MHC class II mismatched model. Transplantation 71: 1146-1153.
20. Raisky, O., B. M. Spriewald, K. J. Morrison, S. Ensminger, T. Mohieddine, J. F. Obadia, M. H. Yacoub, and M. L. Rose. 2003. CD8(+) T cells induce graft vascular occlusion in a CD40 knockout donor/recipient combination. J Heart Lung Transplant 22: 177-183.
21. Womer, K. L., M. H. Sayegh, and H. Auchincloss, Jr. 2001. Involvement of the direct and indirect pathways of allorecognition in tolerance induction. Philos Trans R Soc Lond B Biol Sci 356: 639-647.
22. Rogers, N. J., and R. I. Lechler. 2001. Allorecognition. Am J Transplant 1: 97-102.
23. Poindexter, N., S. Shenoy, T. Howard, M. W. Flye, and T. Mohanakumar. 1997. Allograft infiltrating cytotoxic T lymphocytes recognize kidney-specific human minor histocompatibility antigens. Clin Transplant 11: 174-177.
24. Ouwehand, A. J., C. C. Baan, L. M. Vaessen, N. H. Jutte, A. H. Balk, E. Bos, F. H. Claas, and W. Weimar. 1992. The influence of HLA-mismatches on phenotypic and functional characteristics of graft infiltrating lymphocytes after heart transplantation. Transpl Int 5 Suppl 1: S673-675.
25. Jutte, N. H., M. H. van Batenburg, C. R. Daane, P. Heijse, L. M. Vaessen, A. H. Balk, B. Mochtar, F. H. Claas, and W. Weimar. 1992. Lysis of heart endothelial cells from donor origin by cardiac graft infiltrating cells. Transpl Int 5 Suppl 1: S645-647.
26. Hadley, G. A., C. Charandee, M. R. Weir, D. Wang, S. T. Bartlett, and C. B. Drachenberg. 2001. CD 103+ CTL accumulate within the graft epithelium during clinical renal allograft rejection. Transplantation 72: 1548-1555.
27. Newell, K. A., G. He, Z. Guo, O. Kim, G. L. Szot, I. Rulifson, P. Zhou, J. Hart, J. R. Thistlethwaite, and J. A. Bluestone. 1999. Cutting edge: blockade of the CD28/B7 costimulatory pathway inhibits intestinal allograft rejection mediated by CD4+ but not CD8+ T cells. J Immunol 163: 2358-2362.
28. Trambley, J., A. W. Bingaman, A. Lin, E. T. Elwood, S. Y. Waitze, J. Ha, M. M. Durham, M. Corbascio, S. R. Cowan, T. C. Pearson, and C. P. Larsen. 1999. Asialo GM1(+) CD8(+) T cells play a critical role in costimulation blockade-resistant allograft rejection. J Clin Invest 104: 1715-1722.
29. Paavonen, T., A. Mennander, I. Lautenschlager, S. Mattila, and P. Hayry. 1993. Endothelialitis and accelerated arteriosclerosis in human heart transplant coronaries. J Heart Lung Transplant 12: 117-122.
30. Hasegawa, S., G. Becker, H. Nagano, P. Libby, and R. N. Mitchell. 1998. Pattern of graft- and host-specific MHC class II expression in long-term murine cardiac allografts: origin of inflammatory and vascular wall cells. Am J Pathol 153: 69-79.
31. Derbigny, W. A., M. S. Kerr, and R. M. Johnson. 2005. Pattern recognition molecules activated by Chlamydia muridarum infection of cloned murine oviduct epithelial cell lines. J Immunol 175: 6065-6075.
32. Johnson, R. M. 2004. Murine oviduct epithelial cell cytokine responses to Chlamydia muridarum infection include interleukin-12-p70 secretion. Infect Immun 72: 3951-3960.
33. Johnson, R., D. W. Lancki, and F. W. Fitch. 1993. Accessory molecules involved in antigen-mediated cytolysis and lymphokine production by cytotoxic T lymphocyte subsets. I. Identification of functions for the T cell surface molecules Ly-6C and Thy-1. J Immunol 151: 2986-2999.
34. Nelson, D. E., D. P. Virok, H. Wood, C. Roshick, R. M. Johnson, W. M. Whitmire, D. D. Crane, O, Steele-Mortimer, L. Kari, G. McClarty, and H. D. Caldwell. 2005. Chlamydial IFN-{gamma} immune evasion is linked to host infection tropism. Proc Natl Acad Sci USA 102: 10658-10663.
35. Schulze, D. H., L. R. Pease, S. S. Geier, A. A. Reyes, L. A. Sarmiento, R. B. Wallace, and S. G. Nathenson. 1983. Comparison of the cloned H-2 Kbm1 variant gene with the H-2 Kb gene shows a cluster of seven nucleotide differences. Proc Natl Acad Sci USA 80: 2007-2011.
36. Sprent, J., M. Schaefer, D. Lo, and R. Korngold. 1986. Properties of purified T cell subsets. II. In vivo responses to class I vs. class II H-2 differences. J Exp Med 163: 998-1011
37. Vallera, D. A., P. A. Taylor, J. Sprent, and B. R. Blazar. 1994. The role of host T cell subsets in bone marrow rejection directed to isolated major histocompatibility complex class I versus class II differences of bm1 and bm12 mutant mice. Transplantation 57: 249-256.
38. McIntyre, K. R., and J. G. Seidman. 1984. Nucleotide sequence of mutant I-A beta bm12 gene is evidence for genetic exchange between mouse immune response genes. Nature 308: 551-553.
39. Salomon, R. N., C. C. Hughes, F. J. Schoen, D. D. Payne, J. S. Pober, and P. Libby. 1991. Human coronary transplantation-associated arteriosclerosis. Evidence for a chronic immune reaction to activated graft endothelial cells. Am J Pathol 138: 791-798.
40. Ozdemir, B. H., P. K. Aksoy, A. N. Haberal, B. Demirhan, and M. Haberal. 2004. Relationship of HLA-DR expression to rejection and mononuclear cell infiltration in renal allograft biopsies. Ren Fail 26: 247-251.
41. Marelli-Berg, F. M., and R. I. Lechler. 1999. Antigen presentation by parenchymal cells: a route to peripheral tolerance? Immunol Rev 172: 297-314.
42. Salom, R. N., J. A. Maguire, and W. W. Hancock. 1998. Endothelial activation and cytokine expression in human acute cardiac allograft rejection. Pathology 30: 24-29.
43. Palmer, H., and P. Libby. 1992. Interferon-beta. A potential autocrine regulator of human vascular smooth muscle cell growth. Lab Invest 66: 715-721.
44. Christopher, K., Y. Liang, T. F. Mueller, R. DeFina, H. He, K. J. Haley, M. A. Exley, P. W. Finn, and D. L. Perkins. 2004. Analysis of the major histocompatibility complex in graft rejection revisited by gene expression profiles. Transplantation 78: 788-798.
45. Cercek, M., M. Matsumoto, H. Li, K. Y. Chyu, A. Peter, P. K. Shah, and P. C. Dimayuga. 2006. Autocrine role of vascular IL-15 in intimal thickening. Biochem Biophys Res Commun 339: 618-623.
46. Suzuki, H., K. Eshima, Y. Takagaki, S. Hanaoka, M. Katsuki, M. Yokoyama, T. Hasegawa, S. Yamazaki, and N. Shinohara. 1994. Origin of a T cell clone with a mismatched combination of MHC restriction and coreceptor expression. J Immunol 153: 4496-4507.
47. Shinohara, N., N. Hozumi, M. Watanabe, J. A. Bluestone, R. Johnson-Leva, and D. H. Sachs. 1988. Class II antigen-specific murine cytolytic T lymphocytes (CTL). II. Genuine class II specificity of Lyt-2+ CTL clones. J Immunol 140: 30-36.
48. Watanabe, H., M. Fujiwara, T. Mashiko, and M. Ito. 1989. T-cell clones with L3T4-positive or Lyt-2-positive phenotypes responding to mutant MHC class II antigen and inducing graft versus host reaction. J Invest Dermatol 93: 691-694.
49. Hioe, C. E., and V. S. Hinshaw. 1989. Induction and activity of class II-restricted, Lyt-2+ cytolytic T lymphocytes specific for the influenza H5 hemagglutinin. J Immunol 142: 2482-2488.
50. Morrison, L. A., V. L. Braciale, and T. J. Braciale. 1985. Expression of H-2I region-restricted cytolytic activity by an Lyt-2+ influenza virus-specific T lymphocyte clone. J Immunol 135: 3691-3696.
51. Matechak, E. O., N. Killeen, S. M. Hedrick, and B. J. Fowlkes. 1996. MHC class II-specific T cells can develop in the CD8 lineage when CD4 is absent. Immunity 4: 337-347.
52. Martin, P., A. Duran, S. Minguet, M. L. Gaspar, M. T. Diaz-Meco, P. Rennert, M. Leitges, and J. Moscat. 2002. Role of zeta PKC in B-cell signaling and function. EMBO J. 21: 4049-4057.
53. Hashimoto, A., K. Takeda, M. Inaba, M. Sekimata, T. Kaisho, S. Ikehara, Y. Homma, S. Akira, and T. Kurosaki. 2000. Cutting edge: essential role of phospholipase C-gamma 2 in B cell development and function. J Immunol 165: 1738-1742.
54. Smith-Garvin, J. E., G. A. Koretzky, and M. S. Jordan. 2009. T cell activation. Annu Rev Immunol 27: 591-619.
55. Zheng, Y., H. Liu, J. Coughlin, J. Zheng, L. Li, and J. C. Stone. 2005. Phosphorylation of RasGRP3 on threonine 133 provides a mechanistic link between PKC and Ras signaling systems in B cells. Blood 105: 3648-3654.
56. Kroll, J., X. Shi, A. Caprioli, H. H. Liu, C. Waskow, K. M. Lin, T. Miyazaki, H. R. Rodewald, and T. N. Sato. 2005. The BTB-kelch protein KLHL6 is involved in B-lymphocyte antigen receptor signaling and germinal center formation. Mol Cell Biol 25: 8531-8540.
57. Veldhoen, M., K. Hirota, J. Christensen, A. O'Garra, and B. Stockinger. 2009. Natural agonists for aryl hydrocarbon receptor in culture medium are essential for optimal differentiation of Th17 T cells. J Exp Med 206: 43-49.
58. Svensson, C., A. E. Silverstone, Z. W. Lai, and K. Lundberg. 2002. Dioxin-induced adseverin expression in the mouse thymus is strictly regulated and dependent on the aryl hydrocarbon receptor. Biochem Biophys Res Commun 291: 1194-1200.
59. Vogel, C. F., E. Sciullo, W. Li, P. Wong, G. Lazennec, and F. Matsumura. 2007. RelB, a new partner of aryl hydrocarbon receptor-mediated transcription. Mol Endocrinol 21: 2941-2955.
60. Vogel, C. F., and F. Matsumura. 2009. A new cross-talk between the aryl hydrocarbon receptor and RelB, a member of the NF-kappaB family. Biochem Pharmacol 77: 734-745.
61. Vogel, C. F., D. Wu, S. R. Goth, J. Baek, A. Lollies, R. Domhardt, A. Grindel, and I. N. Pessah. 2013. Aryl hydrocarbon receptor signaling regulates NF-kappaB RelB activation during dendritic-cell differentiation. Immunology and cell biology 91: 568-575.
62. Yuan, X., M. J. Ansari, F. D'Addio, J. Paez-Cortez, I. Schmitt, M. Donnarumma, O. Boenisch, X. Zhao, J. Popoola, M. R. Clarkson, H. Yagita, H. Akiba, G. J. Freeman, J. Iacomini, L. A. Turka, L. H. Glimcher, and M. H. Sayegh. 2009. Targeting Tim-1 to overcome resistance to transplantation tolerance mediated by CD8 T17 cells. Proc Natl Acad Sci USA 106: 10734-10739.
63. Burrell, B. E., K. Csencsits, G. Lu, S. Grabauskiene, and D. K. Bishop. 2008. CD8+Th17 mediate costimulation blockade-resistant allograft rejection in T-bet-deficient mice. J Immunol 181: 3906-3914.
64. Yen, H. R., T. J. Harris, S. Wada, J. F. Grosso, D. Getnet, M. V. Goldberg, K. L. Liang, T. C. Bruno, K. J. Pyle, S. L. Chan, R. A. Anders, C. L. Trimble, A. J. Adler, T. Y. Lin, D. M. Pardoll, C. T. Huang, and C. G. Drake. 2009. Tc17 CD8 T cells: functional plasticity and subset diversity. J Immunol 183: 7161-7168.
65. Sharma, L. C., N. Muirhead, and A. I. Lazarovits. 1997. Human mouse chimeric CD7 monoclonal antibody (SDZCHH380) for the prophylaxis of kidney transplant rejection: analysis beyond 4 years. Transplant Proc 29: 323-324.
66. Sun, Z., C. W. Arendt, W. Ellmeier, E. M. Schaeffer, M. J. Sunshine, L. Gandhi, J. Annes, D. Petrzilka, A. Kupfer, P. L. Schwartzberg, and D. R. Littman. 2000. PKC-theta is required for TCR-induced NF-kappaB activation in mature but not immature T lymphocytes. Nature 404: 402-407.
67. Bauer, B., N. Krumbock, N. Ghaffari-Tabrizi, S. Kampfer, A. Villunger, M. Wilda, H. Hameister, G. Utermann, M. Leitges, F. Uberall, and G. Baier. 2000. T cell expressed PKCtheta demonstrates cell-type selective function. Eur J Immunol 30: 3645-3654.
68. Rott, O., J. Charreire, M. Semichon, G. Bismuth, and E. Cash. 1995. B cell superstimulatory influenza virus (H2-subtype) induces B cell proliferation by a PKC-activating, Ca(2+)-independent mechanism. J Immunol 154: 2092-2103.
69. June, C. H., J. A. Ledbetter, M. M. Gillespie, T. Lindsten, and C. B. Thompson. 1987. T-cell proliferation involving the CD28 pathway is associated with cyclosporine-resistant interleukin 2 gene expression. Mol Cell Biol 7: 4472-4481.
70. Ghosh, P., A. Sica, M. Cippitelli, J. Subleski, R. Lahesmaa, H. A. Young, and N. R. Rice. 1996. Activation of nuclear factor of activated T cells in a cyclosporin A-resistant pathway. J Biol Chem 271: 7700-7704.
71. Ghosh, P., M. A. Buchholz, S. Yano, D. Taub, and D. L. Longo. 2002. Effect of rapamycin on the cyclosporin A-resistant CD28-mediated costimulatory pathway. Blood 99: 4517-4524.
72. Riella, L. V., T. Liu, J. Yang, S. Chock, T. Shimizu, B. Mfarrej, I. Batal, X. Xiao, M. H. Sayegh, and A. Chandraker. 2012. Deleterious effect of CTLA4-Ig on a Treg-dependent transplant model. Am J Transplant 12: 846-855.
73. Yang, J., L. V. Riella, O. Boenisch, J. Popoola, S. Robles, T. Watanabe, V. Vanguri, X. Yuan, I. Guleria, L. A. Turka, M. H. Sayegh, and A. Chandraker. 2009. Paradoxical functions of B7: CD28 costimulation in a MHC class II-mismatched cardiac transplant model. Am J Transplant 9: 2837-2844.
74. Vincenti, F., B. Charpentier, Y. Vanrenterghem, L. Rostaing, B. Bresnahan, P. Darji, P. Massari, G. A. Mondragon-Ramirez, M. Agarwal, G. Di Russo, C. S. Lin, P. Garg, and C. P. Larsen. 2010. A phase III study of belatacept-based immunosuppression regimens versus cyclosporine in renal transplant recipients (BENEFIT study). Am J Transplant 10: 535-546.
75. Pereira, G. M., J. F. Miller, and E. M. Shevach. 1990. Mechanism of action of cyclosporine A in vivo. II. T cell priming in vivo to alloantigen can be mediated by an IL-2-independent cyclosporine A-resistant pathway. J Immunol 144: 2109-2116.

1. A method of identifying a subset of CD8 T cells that is resistant to an inhibitor, the method comprising the step of:
determining whether a subset of CD8 T cells express an elevated level of at least one biomarker selected from the group consisting of Scin and Pla2g4a;
wherein the existence of the elevated level of the at least one biomarker is indicative of the expansion of the CD8 T cells within the subset.

2. The method of claim 1, wherein the inhibitor is selected from the group consisting of cyclosporine, rapamycin and tacrolimus.

3. The method of claim 1, wherein the CD8 T cells of the subset are from a mammal.

4. The method of claim 1, wherein the at least one biomarker is a human biomarker.

5. A method of diagnosing a condition in an individual, the method comprising the step of:
determining if the individual has experienced an expansion of a subset of CD8 T cells that are resistant to an inhibitor selected from the group consisting of cyclosporine, tacrolimus and rapamycin.

6. The method of claim 5, wherein the step of determining if the individual has experienced an expansion of a subset of CD8 T cells comprises the steps of:
measuring the level of expression of at least one biomarker selected from a group consisting of Scin and Plag2a4a in a population of the subset of CD8 T cells from the individual; and
determining if such level of expression is elevated.

7. The method of claim 6, wherein the at least one biomarker is a human biomarker.

8. The method of claim 5, wherein the condition comprises rheumatoid arthritis.

9. The method of claim 5, wherein the condition comprises allograft rejection.

10. A method for treating a condition in an individual comprising the step of:
administering at least one therapeutically effective dose of at least one compound to the individual, the at least one compound capable of inhibiting proliferation of a subset of CD8 T cells from the individual that express aryl hydrocarbon receptor and are resistant to an inhibitor selected from the group consisting of a calcineurin inhibitor and a mTOR inhibitor;
wherein the at least one compound comprises a calcineurin inhibitor and an aryl hydrocarbon receptor inhibitor.

11. The method of claim 10, wherein the condition comprises rheumatoid arthritis and the step of administering at least one therapeutically effective dose is performed to treat the individual experiencing rheumatoid arthritis.

12. The method of claim 10, wherein the condition comprises transplant rejection and the step of administering at least one therapeutically effective dose is performed to treat the individual experiencing allograft vasculopathy or chronic allograft rejection.

13. The method of claim 10, further comprising the step of monitoring the development of the condition in the individual by determining if the individual has experienced an expansion of the subset of CD8 T cells.

14. The method of claim 13, wherein the step of determining if the individual has experienced an expansion of the subset of CD8 T cells comprises the step of:
determining whether the individual has elevated expression of at least one biomarker selected from Scin or Plag2g4a in a population of the subset of CD8 T cells; and
wherein the existence of elevated expression of the at least one biomarker is indicative of the proliferation of CD8 T cells.

15. The method of claim 10, wherein the CD8 T cells of the subset comprise CD8bm12-1 or an equivalent cell type.

16. The method of claim 14, wherein the at least one biomarker comprises a human biomarker.
